(19) United States
(12) Patent Application Publication
(10) Pub. No.: US 2002/0099210 A1
(43) Pub. Date: Jul. 25, 2002

(54) Propanoic acid derivatives as integrin inhibitors

(76) Inventors: Rikki Peter Alexander, High Wycombe (GB); Barry John Langham, Reading (GB); James Thomas Reuberson, Loughborough (GB); Emma Louise Trown, Loughborough (GB); Graham John Warrellow, Northwood (GB)

Correspondence Address: Mackiewicz & Norris LLP, 46th Floor, One Liberty Place, Philadelphia, PA, 19103, US

(21) Appl. No.: 09/738,020

(22) Filed: Dec. 15, 2000

(30) Foreign Application Priority Data
Dec. 17, 1999 (GB) 9929988.5

(51) Int. Cl. C 07D 4 1/02; C07F 9/60; C07F 9/62; C07D 215/12

(52) U.S. Cl. 546/23; 546/139; 546/148; 546/176

(57) ABSTRACT

Propanoic acid derivatives of formula (1) are described:

Ar—X¹—Ar¹—Z—R (1)

in which

Ar is a nitrogen base containing group;

X¹ is a linker atom or group;

Ar¹ is an optionally substituted pyridine or pyridine-N-oxide;

Z is a group —CH(R¹³)CH₂— [in which R¹³ is R^{13a} or Alk^{1a}R^{13a}, R^{13a} is a hydrogen atom or an optionally substituted aliphatic, cycloaliphatic, heteroaliphatic, heterocycloaliphatic, aromatic or heteroaromatic group and Alk^{1a} is an optionally substituted C₁₋₃alkylene chain], —C(R^{12a})(R¹³)—CH(R^{12b})— [in which R^{12a} and R^{12b} together with the carbon atoms to which they are attached form a C₃₋₇cycloalkyl group] or —C(R¹³)═CH—;

R is a carboxylic acid (—CO₂H) or a derivative or biostere thereof;

and the salts, solvates, hydrates and N-oxides thereof.

The compounds are able to inhibit the binding of integrins to their ligands and are of use in the prophylaxis and treatment of immune or inflammatory disorders.

This invention relates to a series of propanoic acid derivatives, to compositions containing them, to processes for their preparation and to their use in medicine.

Over the last few years it has become increasingly clear that the physical interaction of a cell with other cells or components of the extracellular matrix plays an important role in regulating its response to external stimuli such as chemotactic factors, growth factors, cytokines, and inflammatory mediators [Juliano and Haskill, J. Cell Biol. 120, 577-585 (1993); Miyamoto et al J. Cell Biol. 135, 1633-1642 (1996)]. Furthermore, the physical attachment of cells to other cells or surfaces may be crucial for development of some normal physiological responses.

In many disease states normal physiological responses are inappropriately triggered and are detrimental to the well being of the host. Since adhesion molecules play a role in the physical interactions of cells, antagonists of adhesion molecules may be able to inhibit some of the detrimental biological responses found in many disease states.

The adhesion molecules have been sub-divided into different groups on the basis of their structure. One family of adhesion molecules which is believed to play a particularly important role in informing a cell about the nature of its extracellular environment is the integrin family. Members of this family are involved in helping to regulate processes such as proliferation, apoptosis, migration and gene expression in a range of different cell types. They have also been shown to play a key role in regulating immune and inflammatory responses.

The integrin family of cell surface adhesion molecules has a typical non-covalently linked heterodimer structure. At least 16 different integrin alpha chains and 8 different integrin beta chains have been identified [Newman, P. et al, Molecular Medicine Today, 304 (1996)]. The members of the family are typically named according to their heterodimer composition although trivial nomenclature is widespread in this field. Thus the integrin αᵥβ₃ consists of the alpha v chain non-covalently linked to the beta 3 chain.

Some integrin chains are capable of pairing with more than one partner. For example, the alpha v chain has also been reported to pair with the beta 1 chain, the beta 5 chain, the beta 6 chain and the beta 8 chain to give molecules which bind to different sets of ligands and which are referred to respectively as the integrins αᵥβ₁, αᵥβ₅, αᵥβ₆ and αᵥβ₈.

Integrins containing the αᵥ subunit form a family of integrins which generally (but not always) bind to vitronectin although several of them will bind to a range of other matrix molecules and/or cell surface molecules. For example αᵥβ₃ will bind to molecules such as vitronectin, fibronectin, fibrinogen, osteopontin, bone sialoprotein, thrombospondin, pro von Willebrand factor and CD31.

The importance of integrin function in normal physiological responses is highlighted by two human deficiency diseases in which integrin function is defective. Thus, in the disease termed Leukocyte Adhesion Deficiency (LAD) there is a defect in one of the families of integrins expressed on leukocytes. Patients suffering from this diesease show a dramatically reduced ability to recruit leukocytes to inflammatory sites. In the case of patients suffering from the disease termed Glanzman's thrombasthenia (a defect in a member of the beta 3 integrin family) there is a defect in blood clotting.

The interaction of cells with components of the extracellular environment via receptors containing αᵥ has been reported to be involved in a number of cellular responses which may be important in human disease states. These include endothelial cell proliferation and angiogenesis [Friedlander M, et al, Science 270, 1500-1502 (1995)], coronary smooth muscle cell migration, proliferation and extracellular matrix invasion [Panda, D., PNAS, 94, 9308-9313 (1997)], regulation of other integrin molecules on different cell types [Blystone, S D. J. Cell Biol. 127, 1129-1137 (1994); Imhof, B. Eur. J. Immunol, 27, 3242-3252 (1997)] and bone resorption [Ross, F. P. et al, J. Biol. Chem. 268 9901-9907 (1993)]. Furthermore, the αᵥ receptor has been reported to bind to the protease MMP-2 and this may also modify cell function [Brooks, P. C. et a/, Cell, 92, 391400 (1998)].

Monoclonal antibodies and peptides have also been used to demonstrate in animal models that potentially beneficial changes in physiology can be achieved by blocking the function of αᵥ-containing integrin receptors. For example, Mitjans, F. et al [Journal of Cell Science, 108, 2825-2838 (1995)] showed that in a mouse model an antibody that bound to the αᵥ chain inhibited tumour development and metastasis. Brooks P. C., et al; [J. Clin. Invest. 96, 1815-1822 (1995)] demonstrated that an antibody that blocked the function of αᵥβ₃ inhibited the growth of a tumour implanted into a piece of human skin grafted on to a SCID mouse. Christofidou-Solomidou M, [Am. J. Pathol. 151, 975-983 (1997)] has reported that an anti-αᵥ monoclonal antibody inhibited angiogenesis at the site of wound healing. Hammes H-P, et al, [Nature Medicine, 2, 529-533 (1996)] showed that an αᵥ integrin antagonist cyclic peptide inhibited retinal neovascularisation in a model which may have relevance to the human disease states of retinopathy and senile macular degeneration. Srivata S, et al [Cardiovascular Research 36, 408-428 (1997)] have reported that in an animal model a peptidic αᵥβ₃ antagonist can limit neointimal hyperplasia and luminal stenosis.

αᵥβ₃ has been reported to bind to a molecule expressed on endothelial cells termed CD31 [Piali, L. et al, J. Cell Biol. 130, 451-460 (1995)]. Thus αᵥβ₃ may play a role in leukocyte extravasation. It has also been shown to be capable of co-stimulating T-cell degranulation [Ybarrondo, B. Immunology, 91, 186-192 (1997)]. Inhibition of αᵥ function may down regulate immune and/or inflammatory responses.

αᵥβ₃ has also been shown to play a role in the ingestion of apoptotic cells by macrophages [Akbar, A. N. et al, J. Exp. Med 180, 1943-1947 (1994)]. The rapid phagocytosis of apoptotic cells may be a physiological method of reducing inflammatory responses associated with cell lysis. The modulation of αᵥβ₃ function may alter the inflammatory responses mounted in regions of apoptosis. In some disease states this may be beneficial.

It has also been shown that members of the αᵥfamily play a key role in the ability of osteoclasts to resorb bone. An imbalance between bone formation and resorption can lead to major health problems. Blockade of αᵥ containing receptors can inhibit bone resorption in an animal model [Engleman, V. W. et al J. Clin. Invest. 99, 2284-2292, (1997)] and this suggests that αᵥ antagonists may be useful in the treatment of human diseases such as osteoporosis, Paget's disease, humoral hypercalcaemia of malignancy and metastic bone disease.

αᵥ containing receptors are often upregulated at sites of angiogenesis where this occurs for example in tumours, and some pathological conditions. Arap W, et al [Science, 279, 377-380, (1998)] have shown that peptides that bind to αᵥ containing receptors can be used to deliver drugs to such sites and an antibody recognising an αᵥ integrin has been shown to be capable of imaging tumour vasculature [Sipkins, D. A. et al Nature Medicine, 4, 623-626 (1998].

The tissue distribution and range of ligands of different members of the αᵥ integrin family suggests that these molecules may have different physiological roles. This view is supported by Friedlander M et al [ibid] who showed that angiogenesis associated with different growth factors was dependent on different αᵥ containing integrins.

Inhibition of an αᵥ-mediated cell interaction can be expected to be beneficial in a number of disease states. However, because of the ubiquitous distribution and wide range of functions performed by other members of the integrin family it is important to be able to identify selective inhibitors of the αᵥ subgroup.

We have now found a group of compounds which are potent and selective inhibitors of αᵥ integrins. Members of the group are able to inhibit αᵥ integrins such as αᵥβ₃ and/or αᵥβ₅ at concentrations at which they generally have no or minimal inhibitory action on integrins of other subgroups. The compounds are thus of use in medicine, for example in the prophylaxis and treatment of diseases or disorders involving inappropriate growth or migration of cells as described hereinafter.

Thus according to one aspect of the invention we provide a compound of formula (1):

Ar—X¹—Ar¹—Z—R (1)

wherein:

(1) Ar is a group R^{1a}[N(R²)]_{q}L¹Ar²— in which:

R^{1a} is a nitrogen base and q is zero or the integer one;

R² is a hydrogen atom or an optionally substituted aliphatic, heteroaliphatic, cycloaliphatic, polycycloaliphatic, heterocycloaliphatic, heteropolycycloalphatic, aromatic or heteroaromatic group;

L¹ is a covalent bond or a —[C(R³)(R⁴)]ₙ— [where R³ and R⁴, which may be the same or different, is each a hydrogen atom or a straight or branched alkyl group or a hydroxyl group and n is the integer one or two], —C(O)—, —C(S)—, —S(O)—, —S(O)₂—, —P(O)—, —P(O)(OR^{a})— [where R^{a} is a hydrogen atom or a straight or branched C₁₋₆alkyl group] or —P(O)(OR^{a})O— group; and

Ar² is an optionally substituted six-membered 1,4-arylene or 1,4-heteroarylene ring; or

(2) Ar is a bicyclic ring:

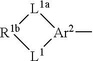

in which R^{1b} is a nitrogen base, L¹ and Ar² are as just defined and —L^{1a}— is a covalent bond a —(CH₂)₃— group or a group L¹ as just defined;

X¹ is an —O— or —S— atom or a group selected from —C(O)—, —C(S)—, —S(O)—, —S(O)₂—, —C(R⁵)(R⁶)— {where R⁵ is a hydrogen atom or an optionally substituted straight or branched alkyl group and R⁶ is a hydrogen or halogen atom or a straight or branched alkyl, haloalkyl, haloalkoxy, alkylthio, aromatic, heteroaromatic, or —(Alk¹ )ₘR⁷ group [in which Alk¹ is a C₁₋₃alkylene chain, m is zero or the integer 1 and R⁷ is a —OH, —SH, —NO₂, —CN, —CO₂H, —CO₂R⁸ (where R⁸ is an optionally substituted straight or branched C₁₋₆alkyl group), —OR⁸, —SO₃H, —SOR⁸, —SO₂R⁸, —SO₃R⁸, —OCO₂R⁸, —C(O)H, —C(O)R⁸, —OC(O)R⁸, —C(S)R⁸, —NR⁹R¹⁰ (where R⁹ and R¹⁰, which may be the same or different is each a hydrogen atom or a straight or branched alkyl group), —C(O)N(R⁹)(R¹⁰), —OC(O)N(R⁹)(R¹⁰), —N(R⁹)C(O)R¹⁰, —CSN(R⁹)(R¹⁰), —N(R⁹)C(S)R¹⁰, —SO₂N(R⁹)(R¹⁰), —N(R⁹)SO₂R¹⁰, —N(R⁹)C(O)N(R¹⁰)(R¹¹) [where R¹¹ is a hydrogen atom or a straight or branched alkyl group], —N(R⁹)C(S)N(R¹⁰)(R¹¹), —N(R⁹)SO₂N(R¹⁰)(R¹¹), aromatic or hetero-aromatic group]}, —C(═NOH)—, —C(═CR⁵R⁶)— or —N(R⁵)—;

Z is a group —CH(R¹³)CH₂— [in which R¹³ is R^{13a} or Alk^{1a}R^{13a}, R^{13a} is a hydrogen atom or an optionally substituted aliphatic, cycloaliphatic, heteroaliphatic, heterocycloaliphatic, aromatic or heteroaromatic group, and Alk^{1a} is a C₁₋₃alkylene chain optionally substituted with one, two, three or more halogen atoms or straight or branched alkyl, haloalkyl, alkoxy, haloalkoxy, alkylthio, aromatic or heteroaromatic groups], —C(R^{12a})(R¹³)—CH(R^{12b})— [in which R^{12a} and R^{12b} together with the carbon atoms to which they are attached form a C₃₋₇cycloalkyl group] or —C(R¹³)═CH—;

R is a carboxylic acid (—CO₂H) or a derivative or biostere thereof;

Ar¹ is an optionally substituted heterocycle of formula

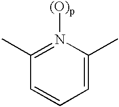

wherein p is zero or the integer 1;

and the salts, solvates, hydrates and N-oxides thereof.

It will be appreciated that when p is the integer 1 O exists as O⁻ and N exists as N⁺ in the heterocycle Ar¹.

It will be appreciated that certain compounds of formula (1) may exist as geometric isomers (E or Z isomers). The compounds may also have one or more chiral centres, and exist as enantiomers or diastereomers. The invention is to be understood to extend to all such geometric isomers, enantiomers, diastereomers and mixtures thereof, including racemates. Formula (1) and the formulae hereinafter are intended to represent all individual isomers and mixtures thereof, unless stated or shown otherwise. In addition, compounds of formula (1) may exist as tautomers, for example keto (CH₂C═O)-enol (CH═CHOH) tautomers. Formula (1) and the formulae hereinafter are intended to repressent all individual tautomers and mixtures thereof, unless stated otherwise.

In the compounds of the invention as represented by formula (1) and the more detailed description hereinafter certain of the general terms used in relation to substituents are to be understood to include the following atoms or groups unless specified otherwise.

Thus as used herein the term “optionally substituted straight or branched alkyl”, whether present as a group or part of a group includes straight or branched C₁₋₆alkyl groups, for example C₁₋₄alkyl groups such as methyl, ethyl, n-propyl, i-propyl or t-butyl groups. Similarly, the terms “optionally substituted straight or branched alkenyl” or “optionally substituted straight or branched alkynyl” are intended to mean C₂₋₆alkenyl or C₂₋₆alkynyl groups such as C₂₋₄alkenyl or C₂₋₄alkynyl groups. Optional substituents present on those groups include those optional substituents mentioned hereinafter in relation to R² optionally substituted aliphatic groups.

The term “halogen atom” is intended to include fluorine, chlorine, bromine or iodine atoms.

The term “straight or branched haloalkyl” is intended to include the alkyl groups just mentioned substituted by one, two or three of the halogen atoms just described. Particular examples of such groups include —CF₃, —CCl₃, —CHF₂— —CHCl₂, —CH₂F, and —CH₂Cl groups.

The term “straight or branched alkoxy” as used herein is intended to include straight or branched C₁₋₆alkoxy e.g. C₁₋₄alkoxy such as methoxy, ethoxy, n-propoxy, i-propoxy and t-butoxy. “Haloalkoxy” as used herein includes any of those alkoxy groups substituent by one, two or three halogen atoms as described above. Particular examples include —OCF₃, —OCCl₃, —OCHF₂, —OCHCl₂, —OCH₂F and —OCH₂Cl groups.

As used herein the term “straight or branched alkylthio” is intended to include straight or branched C₁₋₆alkylthio, e.g. C₁₋₄alkylthio such as methylthio or ethylthio groups.

The terms “aromatic” or heteroaromatic” are intended to include those optionally substituted aromatic or heteroaromatic groups described generally and particularly hereinafter in relation to the groups R², R¹⁴, R¹⁵ and R¹⁶.

Where the term “1,4-arylene” is used in relation to Ar² in the formulae herein this is to be understood to mean a ring:

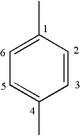

in which the carbon atoms at the one and four positions are attached to the remainder of the molecule. The term “1,4-heteroarylene” is to be understood to mean an equivalent ring structure in which one or more of the carbon atoms at the 2-, 3-, 5- and/or 6-positions of the 1,4-arylene ring is replaced by a nitrogen atom. Examples of 1,4-heteroarylenes include 2,5-pyridyl, 2,5-pyrimidinyl, 2,5-pyrazinyl and 3,6-pyridazinyl groups.

Such arylene and heteroarylene rings may be optionally substituted, each substituent being attached to a carbon atom, where present, at the 2-, 3-, 5- and/or 6-positions. Particular substituents include halogen atoms, or straight or branched alkyl, haloalkyl, alkoxy, haloalkoxy or alkylthio groups, or —OH, —CO₂H, —CO₂R⁸ [where R⁸ is as previously defined], —CN, —NH₂, —NO₂, or straight or branched alkylamino (—NHR⁸) or dialkylamino (—N(R⁸)₂) groups. Where two R⁸ groups are present in such optional substituents these groups may be the same or different.

Nitrogen bases represented by the group R^{1a} in compounds of the invention include acyclic or cyclic nitrogen bases containing one, two, three or more nitrogen atoms. Such bases will generally include one or more carbon atoms and optionally one or more other heteroatoms such as oxygen or suphur atoms.

Particular examples of acyclic nitrogen bases represented by the group R^{1a} include those wherein R^{1a} is a R¹⁴R¹⁵NC(X²)—, R¹⁵C(═NR¹⁴)—, or R¹⁴N═CH— group, in which X² is a ═NR¹⁶, ═O, ═NCN, ═NC(O)NH₂ or ═S group, and each of R¹⁴, R¹⁵ and R¹⁶, which may be the same or different, is a hydrogen atom or an optionally substituted aliphatic, heteroaliphatic, cycloaliphatic, polycycloaliphatic, heterocycloaliphatic, heteropolycycloaliphatic, aromatic or heteroaromatic group.

Optionally substituted aliphatic groups represented by R², R¹⁴, R¹⁵ and/or R¹⁶ include optionally substituted straight or branched C₁₋₁₀ alkyl, e.g. C₁₋₆alkyl, C₂₋₁₀alkenyl e.g. C₂₋₆alkenyl or C₂₋₁₀alkynyl e.g. C₂₋₆alkynyl groups.

Heteroaliphatic groups represented by R², R¹⁴, R¹⁵ and/or R¹⁶ include the aliphatic groups just described but with each group additionally containing one, two, three or four heteroatoms or heteroatom-containing groups. Particular heteroatoms or groups include atoms or groups L² where L² is a linker atom or group. Each L² atom or group may interrupt the aliphatic group, or may be positioned at its terminal carbon atom to connect the group to an adjoining atom or group. Particular examples of suitable L² atoms or groups include —O— or —S— atoms or —C(O)—, —C(O)O—, —C(S)—, —S(O), —S(O)₂—, —N(R¹⁷)— [where R¹⁷ is a hydrogen atom or an optionally substituted straight or branched alkyl group], —N(R¹⁷)O—, —N(R¹⁷)N(R¹⁷)—, —CON(R¹⁷)—, —OC(O)N(R¹⁷)—, —CSN(R¹⁷)—, —N(R¹⁷)CO—, —N(R¹⁷)C(O)O—, —N(R¹⁷)CS—, —S(O)₂N(R¹⁷)—, —N(R¹⁷)S(O)2—, —N(R¹⁷)CON(R¹⁷)—, —N(R¹⁷)CSN(R¹⁷)—, or —N(R¹⁷)SO₂N(R¹⁷)— groups. Where the linker group contains two R¹⁷ substituents, these may be the same or different.

Particular examples of aliphatic groups represented by R², R¹⁴, R¹⁵ and/or R¹⁶ include optionally substituted —CH₃, —CH₂CH₃, —CH(CH₃)₂, —(CH₂)₂CH₃, —(CH₂)₃CH₃, —CH(CH₃)CH₂CH₃, —CH₂CH(CH₃)₂, —C(CH₃)₂, —(CH₂)₄CH₃, —(CH₂)₅CH₃, —CHCH₂, —CHCHCH₃, —CH₂CHCH₂, —CHCHCH₂CH₃, —CH₂CHCHCH₃, —(CH₂)₂CHCH₂, —CCH, —CCCH₃, —CH₂CCH, —CCCH₂CH₃, —CH₂CCCH₃, or —(CH₂)₂CCH groups. Where appropriate each of said groups may be optionally interrupted by one, two, three or more atoms and/or groups L² to form an optionally substituted heteroaliphatic group. Particular examples include optionally substituted —L²CH₃, —CH₂L²CH₃, —L²CH₂CH₃, —CH₂L²CH₂CH₃, —L²CH₂L²CH₃, —(CH₂)₂L²CH₃, —L²(CH₂)₂CH₃— and —(CH₂)₂L²CH₂CH₃ groups.

The optional substituents which may be present on aliphatic or heteroaliphatic groups represented by R², R¹⁴, R¹⁵, and/or R¹⁶ include one, two, three or more substituents where each substituent may be the same or different and is selected from halogen atoms, or C₁₋₆alkoxy, hydroxy, thiol, C₁₋₆alkylthio, optionally substituted C₆₋₁₂arylamino, substituted amino, —CN, —CO₂H, —CO₂R^{8a} (where R^{8a} is an optionally substituted straight or branched C₁₋₆alkyl group), —SO₃H, —SOR^{8a}, —SO₂R^{8a}, —SO₃R^{8a}, —OCO₂R^{8a}, —C(O)H, —C(O)R^{8a}, —OC(O)R^{8a}, —C(S)R^{8a}, —C(O)N(R^{9a})(R^{10a}) (where R^{9a} and R^{10a}, which may be the same or different is each a hydrogen atom or an optionally substituted straight or branched alkyl group), —OC(O)N(R^{9a})(R^{10a}), —N(R⁹a)C(O)R^{10a}, —CSN(R^{9a})(R^{10a}), —N(R^{9a})C(S)(R^{10a}), SO₂N(R^{9a})(R^{10a}), —N(R^{9a})SO₂R^{10a}, —N(R^{9a})C(O)N(R^{10a})(R^{11a}) (where R^{11a} is a hydrogen atom or an optionally substituted straight or branched alkyl group), —N(R^{9a})C(S)N(R^{10a})(R^{11a}), —N(R^{9a})SO₂N(R^{10a})(R^{11a}), or optionally substituted aromatic or heteroaromatic groups. Substituted amino groups include —NHR¹⁸ and —N(R¹⁸)₂ groups where R¹⁸ is a straight or branched alkyl group. Where two R¹⁸ groups are present these may be the same or different. Particular examples of substituted groups represented by R², R¹⁴, R¹⁵ and/or R¹⁶ include those specific groups just described substituted by one, two, or three halogen atoms such as fluorine atoms, for example groups of the type —CH₂CF₃, —CH(CF₃)₂, —CH₂CH₂CF₃, —CH₂CH(CF₃)₂ and —C(CF₃)₂CH₃, or substituted by one or two optionally substituted aromatic or heteroaromatic groups, for example optionally substituted phenyl, pyridinyl or pyrimidinyl groups. Examples of R², R¹⁴, R¹⁵ and/or R¹⁶ groups of this type include optionally substituted benzyl, pyridinylCH₂—, pyrimidinylCH₂ and phenylethyl groups. Optional substituents on these aromatic or heteroaromatic containing groups include those substituents described hereinafter in relation to R² aromatic and heteroaromatic groups.

Optionally substituted cycloaliphatic groups represented by R², R¹⁴, R¹⁵ and/or R¹⁶ include optionally substituted C₃₋₁₀ cycloaliphatic groups. Particular examples include optionally substituted C₃₋₁₀ cycloalkyl, e.g. C₃₋₇ cycloalkyl or C₃₋₁₀ cycloalkenyl, e.g C₃₋₇ cycloalkenyl groups.

Optionally substituted heterocycloaliphatic groups represented by R², R¹⁴, R¹⁵ and/or R¹⁶ include optionally substituted C₃₋₁₀heterocycloaliphatic groups. Particular examples include optionally substituted C₃₋₁₀heterocycloalkyl, e.g. C₃₋₇heterocycloalkyl, or C₃₋₁₀heterocycloalkenyl, e.g. C₃₋₇ hetercycloalkenyl groups, each of said groups containing one, two, three or four heteroatoms or heteroatom-containing groups L² as just defined.

Optionally substituted polycycloaliphatic groups represented by R², R¹⁴, R¹⁵ and/or R¹⁶ include optionally substitued C₇₋₁₀ bi- or tricycloalkyl or C₇₋₁₀bi- or tricycloalkenyl groups. Optionally substituted heteropolycycloaliphatic groups represented by R², R¹⁴, R¹⁵ and/or R¹⁶ include the optionally substituted polycycloaliphatic groups just described, but with each group additionally containing one, two, three or four L² atoms or groups.

Particular examples of R², R¹⁴, R¹⁵ and/or R¹⁶ cycloaliphatic, polycycloaliphatic, heterocycloaliphatic and heteropolycycloaliphatic groups include optionally substituted cyclopropyl, cyclobutyl, cyclopentyl, cyclohexyl, cycloheptyl, 2-cyclobuten-1-yl, 2-cyclopenten-1-yl, 3-cyclopenten-1-yl, adamantyl, norbornyl, norbornenyl, tetrahydrofuranyl, pyrroline, e.g. 2- or 3-pyrrolinyl, pyrrolidinyl, pyrrolidinone, oxazolidinyl, oxazolidinone, dioxolanyl, e.g. 1,3-dioxolanyl, imidazolinyl, e.g. 2-imidazolinyl, imidazolidinyl, pyrazolinyl, e.g. 2-pyrazolinyl, pyrazolidinyl, thiazolinyl, thiazolidinyl, pyranyl, e.g. 2- or 4-pyranyl, piperidinyl, homopiperidinyl, heptamethyleneiminyl, piperidinone, tetrahydropyrimidinyl e.g. 1,4,5,6-tetrahydropyrimidinyl, 1,4-dioxanyl, morpholinyl, morpholinone, 1,4-dithianyl, thiomorpholinyl, piperazinyl, homopiperazinyl, 1,3,5-trithianyl, oxazinyl, e.g. 2H-1,3-, 6H-1,3-, 6H-1,2-, 2H-1,2- or 4H-1,4-oxazinyl, 1,2,5-oxathiazinyl, isoxazinyl, e.g. o- or p-isoxazinyl, oxathiazinyl, e.g. 1,2,5 or 1,2,6-oxathiazinyl, or 1,3,5,-oxadiazinyl groups.

The optional substituents which may be present on the R², R¹⁴, R¹⁵ and R¹⁶ cycloaliphatic, polycycloaliphatic, heterocycloaliphatic or heteropolycycloaliphatic groups include one, two, three or more of those substituents described above in relation to R² aliphatic or heteroaliphatic groups.

Optionally substituted aromatic groups represented by the groups R², R¹⁴, R¹⁵ and/or R¹⁶ include for example monocyclic or bicyclic fused ring C₆₋₁₂ aromatic groups, such as phenyl, 1- or 2-naphthyl, 1- or 2-tetrahydronaphthyl, indanyl or indenyl groups. Each of these aromatic groups may be optionally substituted by one, two, three or more R¹⁹ atoms or groups as defined below.

Heteroaromatic groups represented by the groups R², R¹⁴, R¹⁵ and/or R¹⁶ include for example C₁₋₉ heteroaromatic groups containing for example one, two, three or four heteroatoms selected from oxygen, sulphur or nitrogen atoms. In general, the heteroaromatic groups may be for example monocyclic or bicyclic fused ring heteroaromatic groups. Monocyclic heteroaromatic groups include for example five- or six-membered heteroaromatic groups containing one, two, three or four heteroatoms selected from oxygen, sulphur or nitrogen atoms. Bicyclic heteroaromatic groups include for example eight- to thirteen-membered fused-ring heteroaromatic groups containing one, two or more heteroatoms selected from oxygen, sulphur or nitrogen atoms.

Particular examples of heteroaromatic groups of these types include pyrrolyl, furyl, thienyl, imidazolyl, N—C₁₋₆alkylimidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-thiadiazole, pyridyl, pyrimidinyl, pyridazinyl, pyrazinyl, 1,3,5-triazinyl, 1,2,4-triazinyl, 1,2,3-triazinyl, benzofuryl, [2,3-dihydro]benzofuryl, benzothienyl, benzotriazolyl, indolyl, indolinyl, isoindolyl, indazolinyl, benzimidazolyl, imidazo[1,2-a]pyridyl, benzothiazolyl, benzoxazolyl, benzisoxazolyl, benzopyranyl, [3,4-dihydro]benzopyranyl, quinazolinyl, qunoxalinyl, naphthyridinyl, 5, 6, 7, 8-tetrahydronaphthyridinyl, pyrido[3,4-b]pyridyl, pyrido[3,2-b]pyridyl, pyrido[4,3-b]-pyridyl, quinolinyl, isoquinolinyl, phthalazinyl, tetrazolyl, 5,6,7,8-tetrahydroquinolinyl, 5,6,7,8-tetrahydroisoquinolinyl, and imidyl, e.g. succinimidyl, phthalimidyl, or naphthalimidyl such as 1,8-naphthalimidyl.

Optional substituents which may be present on the aromatic or heteroaromatic groups represented by the groups R², R¹⁴, R¹⁵ and/or R¹⁶ include one, two, three or more substituents, each selected from an atom or group R¹⁹ in which R¹⁹ is —R^{19a} or —Alk³(R^{19a})ₘ, where R^{19a} is a halogen atom, or an amino (—NH₂), substituted amino, nitro, cyano, amidino, hydroxyl (—OH), substituted hydroxyl, formyl, carboxyl (—CO₂H), esterified carboxyl, thiol (—SH), substituted thiol, —COR²⁰ [where R²⁰ is an —Alk³(R^{19a})ₘ, aryl or heteroaryl group], —CSR²⁰, —SO₃H, —SOR²⁰, —SO₂R²⁰, —SO₃R²⁰, —SO₂NH₂, —SO₂NHR²⁰, —SO₂N(R²⁰)₂, —CONH₂, —CSNH₂, —CONHR²⁰, —CSNHR²⁰, —CON[R²⁰]₂, —CSN(R²⁰)₂, —N(R²¹)SO₂R²⁰, [where R²¹ is a hydrogen atom or a straight or branched alkyl group] —N(SO₂R²⁰)₂, —N(R²¹)SO₂NH₂, —N(R²¹)SO₂NHR²⁰, —N(R²¹)SO₂N(R²⁰)₂, —N(R²¹ )COR²⁰, —N(R²¹ )CONH₂, —N(R²¹ )CONHR²⁰, —N(R²¹ )CON(R²⁰)₂, —N(R²¹)CSNH₂, —N(R²¹)CSNHR²⁰, —N(R²¹)CSN(R²⁰)₂, —N(R²¹)CSR²⁰, —N(R²¹)C(O)OR²⁰, —SO₂NHet¹ [where —NHet¹ is an optionally substituted C₅₋₇cyclicamino group optionally containing one or more other —O— or —S— atoms or —N(R²¹)—, —C(O)— or —C(S)— groups], —CONHet¹, —CSNHet¹, —N(R²¹)SO₂NHet¹, —N(R²¹ )CONHet¹, —N(R²¹)CSNHet¹, —SO₂N(R²¹ )Het² [where Het² is an optionally substituted monocyclic C₅₋₇carbocyclic group optionally containing one or more —O— or —S— atoms or —N(R²¹)—, —C(O)— or —C(S)— groups], —Het², —CON(R²¹)Het², —CSN(R²¹)Het², —N(R²¹)CON(R²¹)Het², —N(R²¹)CSN(R²¹)Het², aryl or heteroaryl group; Alk³ is a straight or branched C₁₋₆alkylene, C₂₋₆alkenylene or C₂₋₆alkynylene chain, optionally interrupted by one, two or three —O— or —S— atoms or —S(O)ₙ [where n is an integer 1 or 2] or —N(R²¹)— groups; and m is zero or an integer 1, 2 or 3. It will be appreciated that when two R²⁰ or R²¹ groups are present in one of the above substituents, the R²⁰ or R²¹ groups may be the same or different.

When in the group —Alk³(R^{19a})ₘ m is an integer 1, 2 or 3, it is to be understood that the substituent or substituents R^{19a} may be present on any suitable carbon atom in —Alk³. Where more than one R^{19a} substituent is present these may be the same or different and may be present on the same or different atom in —Alk³. Clearly, when m is zero and no substituent R^{19a} is present the alkylene, alkenylene or alkynylene chain represented by Alk³ becomes an alkyl, alkenyl or alkynyl group.

When R^{19a} is a halogen atom it may be for example a fluorine, chlorine, bromine or iodine atom.

When R^{19a} is a substituted amino group it may be for example a group —NHR²⁰ [where R²⁰ is as defined above] or a group —N(R²⁰)₂ wherein each R²⁰ group is the same or different.

When R^{19a} is a substituted hydroxyl or substituted thiol group it may be for example a group —OR²⁰ or a —SR²⁰ or —SC(═NH)NH₂ group respectively.

Esterified carboxyl groups represented by the group R^{19a} include groups of formula —CO₂Alk⁴ wherein Alk⁴ is a straight or branched, optionally substituted C₁₋₈alkyl group such as a methyl, ethyl, n-propyl, i-propyl, n-butyl, i-butyl, s-butyl or t-butyl group; a C₆₋₁₂arylC₁₋₈alkyl group such as an optionally substituted benzyl, phenylethyl, phenylpropyl, 1-naphthylmethyl or 2-naphthylmethyl group; a C₆₋₁₂aryl group such as an optionally substituted phenyl, 1-naphthyl or 2-naphthyl group; a C₆₋₁₂aryloxyC₁₋₈alkyl group such as an optionally substituted phenyloxymethyl, phenyloxyethyl, 1-naphthyloxymethyl, or 2-naphthyloxymethyl group; an optionally substituted C₁₋₈alkanoyloxyC₁₋₈alkyl group, such as a pivaloyloxymethyl, propionyloxyethyl or propionyloxypropyl group; or a C₆₋₁₂aroyloxyC₁₋₈alkyl group such as an optionally substituted benzoyloxyethyl or benzoyloxy-propyl group. Optional substituents present on the Alk⁴ group include R^{19a} substituents described above.

When Alk³ is present in or as a substituent it may be for example a methylene, ethylene, n-propylene, i-propylene, n-butylene, i-butylene, s-butylene, t-butylene, ethenylene, 2-propenylene, 2-butenylene, 3-butenylene, ethynylene, 2-propynylene, 2-butynylene or 3-butynylene chain, optionally interrupted by one, two, or three —O— or —S—, atoms or —S(O)—, —S(O)₂— or —N(R²¹)— groups.

Aryl or heteroaryl groups represented by the groups R^{19a} or R²⁰ include mono- or bicyclic optionally substituted C₆₋₁₂ aromatic or C₁₋₉ heteroaromatic groups as described above for the group R². The aromatic and heteroaromatic groups may be attached to the remainder of the compound of formula (1) by any carbon or hetero e.g. nitrogen atom as appropriate.

When —NHet¹ or —Het² forms part of a substituent R¹⁹ each may be for example an optionally substituted 2- or 3-pyrrolinyl, pyrrolidinyl, pyrazolinyl, pyrazolidinyl, piperazinyl, imidazolinyl, imidazolidinyl, morpholinyl, thiomorpholinyl, piperidinyl, oxazolidinyl or thiazolidinyl group. Additionally Het² may represent for example, an optionally substituted cyclopentyl or cyclohexyl group. Optional substituents which may be present on —NHet¹ or —Het² include those substituents described above in relation to R⁶.

Particularly useful atoms or groups represented by R¹⁹ include fluorine, chlorine, bromine or iodine atoms, or C₁₋₆alkyl, e.g. methyl, ethyl, n-propyl, i-propyl, n-butyl or t-butyl, optionally substituted phenyl, pyridyl, pyrimidinyl, pyrrolyl, furyl, thiazolyl, thienyl, morpholinyl, thiomorpholinyl, piperazinyl, pyrrolidinyl or piperidinyl, C₁₋₆hydroxyalkyl, e.g. hydroxymethyl or hydroxyethyl, carboxyC₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, optionally substituted phenoxy, pyridyloxy, thiazolyoxy, phenylthio or pyridylthio, C₅₋₇cycloalkoxy, e.g. cyclopentyloxy, haloC₁₋₆alkyl, e.g. trifluoromethyl, haloC₁₋₆alkoxy, e.g. trifluoromethoxy, C₁₋₆alkylamino, e.g. methylamino or ethylamino, amino (—NH₂), aminoC₁₋₆alkyl, e.g. aminomethyl or aminoethyl, C₁₋₆dialkylamino, e.g. dimethylamino or diethylamino, aminoC₁₋₆alkylamino e.g. aminoethyl-amino, Het¹NC₁₋₆alkylamino e.g. morpholinopropylamino, C₁₋₆alkyl-aminoC₁₋₆alkyl, e.g. ethylaminoethyl, C₁₋₆dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethylaminoethoxy, diisopropylaminoethoxy, or dimethylaminopropoxy, hydroxyC₁₋₆alkylamino e.g. hydroxyethylamino, imido, such as phthalimido or naphthalimido, e.g. 1,8-naphthalimido, nitro, cyano, amidino, hydroxyl (—OH), formyl [HC(O)—], carboxyl (—CO₂H), —CO₂Alk⁴ [where Alk⁴ is as defined above], C₁₋₆ alkanoyl e.g. acetyl, optionally substituted benzoyl, thiol (—SH), thioC₁₋₆alkyl, e.g. thiomethyl or thioethyl, —SC(═NH)NH₂, sulphonyl (—SO₃H), —SO₃R²⁰, C₁₋₆alkylsulphinyl e.g. methylsulphinyl, C₁₋₆alkylsulphonyl, e.g. methylsulphonyl, amino-sulphonyl (—SO₂NH₂), C₁₋₆alkylaminosulphonyl, e.g. methylaminosulphonyl or ethylaminosulphonyl, C₁₋₆dialkylaminosulphonyl, e.g. dimethylaminosulphonyl or diethylaminosulphonyl, optionally substituted phenylaminosulphonyl, carboxamido (—CONH₂), C₁₋₆alkylaminocarbonyl, e.g. methylaminocarbonyl or ethylaminocarbonyl, C₁₋₆dialkylaminocarbonyl, e.g. dimethylaminocarbonyl or diethylaminocarbonyl, aminoC₁₋₆alkylaminocarbonyl, e.g. aminoethylaminocarbonyl, C₁₋₆dialkylaminoC₁₋₆alkylaminocarbonyl, e.g. diethylaminoethylaminocarbonyl, aminocarbonylamino, C₁₋₆alkylaminocarbonylamino, e.g. methylaminocarbonylamino or ethylaminocarbonylamino, C₁₋₆dialkylaminocarbonylamino, e.g. dimethylaminocarbonylamino or diethylaminocarbonylamino, C₁₋₆alkylaminocabonylC₁₋₆alkylamino, e.g. methylaminocarbonylmethylamino, aminothiocarbonylamino, C₁₋₆alkylaminothiocarbonylamino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₆alkylamino, e.g. ethylaminothiocarbonylmethylamino, —CONHC(═NH)NH₂, C₁₋₆alkylsulphonylamino, e.g. methylsulphonylamino or ethylsulphonylamino, C₁₋₆dialkylsulphonylamino, e.g. dimethylsulphonylamino or diethylsulphonylamino, optionally substituted phenylsulphonylamino, aminosulphonylamino (—NHSO₂NH₂), C₁₋₆alkylaminosulphonylamino, e.g. methylaminosulphonylamino or ethylaminosulphonylamino, C₁₋₆dialkylaminosulphonylamino, e.g. dimethylaminosulphonylamino or diethylaminosulphonylamino, optionally substituted morpholinesulphonylamino or morpholinesulphonylC₁₋₆alkylamino, optionally substituted phenylaminosulphonylamino, C₁₋₆alkanoylamino, e.g. acetylamino, aminoC₁₋₆alkanoylamino e.g. aminoacetylamino, C₁₋₆dialkylaminoC₁₋₆alkanoylamino, e.g. dimethylaminoacetylamino, C₁₋₆alkanoylaminoC₁₋₆alkyl, e.g. acetylaminomethyl, C₁₋₆alkanoylaminoC₁₋₆alkylamino, e.g. acetamidoethylamino, C₁₋₆alkoxycarbonylamino, e.g. methoxycarbonylamino, ethoxycarbonylamino or t-butoxycarbonylamino or optionally substituted benzyloxy, benzylamino, pyridylmethoxy, thiazolylmethoxy, benzyloxycarbonylamino, benzyloxycarbonylaminoC₁₋₆alkyl e.g. benzyloxycarbonylaminoethyl, thiobenzyl, pyridylmethylthio or thiazolylmethylthio groups.

Where desired, two R¹⁹ substituents may be linked together to form a cyclic group such as a cyclic ether, e.g. a C₁₋₆alkylenedioxy group such as methylenedioxy or ethylenedioxy.

It will be appreciated that where two or more R¹⁹ substituents are present, these need not necessarily be the same atoms and/or groups. In general, the substituent(s) may be present at any available ring position in the aromatic or heteroaromatic group represented by R², R¹⁴, R¹⁵ and/or R¹⁶.

Particular examples of cyclic nitrogen bases represented by the group R^{1a} in compounds of the invention include those wherein R^{1a} is an optionally substituted four- to ten-membered, for example five- to ten-membered, mono- or bicyclic fused-ring heterocycloaliphatic or heteroaromatic group containing one, two, three or more nitrogen atoms and optionally one or more other heteroatoms such as oxygen and sulphur atoms. Particular examples of heterocycloaliphatic groups include five- to seven-membered heterocycloaliphatic groups, particularly five- and six-membered heterocycloaliphatic groups. Particular examples of heteroaromatic groups include five- and six-membered monocyclic heteroaromatic groups and nine- and ten-membered bicyclic heteroaromatic groups. Suitable examples include optionally substituted pyrrolidinyl, pyrrolinyl, piperidinyl, homopiperidinyl, heptamethyleneiminyl, tetrahydropyridinyl, piperazinyl, homopiperazinyl, triazinyl, morpholinyl, thiomorpholinyl, thiazolidinyl, indolinyl, tetrahydroquinolinyl, tetrahydroisoquinolinyl, tetrahydropyrimidinyl e.g. 1,4,5,6-tetrahydropyrimidin-2-yl, tetrahydro-[1,8]-naphthyridinyl e.g. 5,6,7,8-tetrahydro-[1,8]-naphthyridin-2-yl, pyrrolyl, pyrazolyl, imidazolyl, imidazolinyl, imidazolidinyl, triazolyl, tetrazolyl, isoxazolyl, oxazolyl, thiazolyl, isothiazolyl, oxadiazolyl, thiadiazolyl, indolyl, indazolyl, benzimidazolyl, benzoxazolyl, [1,8]-naphthyridinyl, benzisoxazolyl, benzothiazolyl, pyridyl, pyrimidinyl, pyrazinyl, triazinyl, quinolinyl and isoquinolinyl groups. Optional substituents which may be present on these groups include one, two or three of those R¹⁹ substituents described herein. The ring R^{1a} will generally be attached to the —N(R²)— group, L¹ or Ar² as the case may be through any available ring carbon or nitrogen atom.

When in the compounds of the invention the Ar group is a bicyclic ring it may be for example a ring of formula:

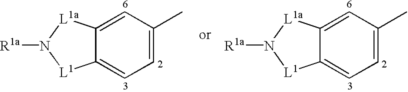

[where R^{1a}N and R^{1a}CH form the nitrogen base R^{1b} described in formula (1)] in which each of the carbon atoms at positions 2-, 3- and 6- may optionally be substituted or replaced by a nitrogen atom as described above in relation to the ring Ar². In these compounds R^{1a}, L¹ and L^{1a} may be as decribed previously. L^{1a} may in particular be a —CH₂—, —(CH₂)₂— or —(CH₂)₃ chain.

When in the compounds of the invention the group Z contains a group R¹³ which is an optionally substituted aliphatic, cycloaliphatic, heteroaliphatic, heterocycloalphatic, aromatic or heteroaromatic group, each of these groups may be any of those previously generally and particularly described for the group R². Optional substituents which may be present on such groups include those described for R², for example one, two or three R¹⁹ substituents as described above when R³ is an aromatic or heteroaromatic group.

When the group Z is —C(R^{12a})(R¹³)—CH(R^{12b}), then R^{12a} and R^{12b} together with the carbon atoms to which they are attached may form for example a cyclopropyl group.

Derivatives of the carboxylic acid group R in compounds of the invention include caboxylic acid esters and amides. Particular esters and amides include —CO₂Alk⁴ and —CONR⁹R¹⁰ groups as described herein. Biosteres of the carboxylic acid group R include tetrazoles, or other acids such as squaric acid, phosphoric acid, sulphonic acid, sulphinic acid, or boronic acid.

In pyridines and pyridine N-oxides represented by Ar¹ when a carbon atom is available, it may be optionally substituted by a halogen atom or a straight or branched C₁₋₆alkyl, haloalkyl, alkoxy, haloalkoxy or alkylthio group, or a —OH, —CO₂H, —CO₂R⁸, —CN, —NH₂, —NO₂ or straight or branched alkylamino or dialkylamino group.

When the optional substituent on Ar¹ is a straight or branched alkylamino group it may be for example a group —NHR²² [where R²² is a straight or branched C₁₋₆alkyl group] and when the optional substituent is a straight or branched dialkylamino group it may be for example a group —N(R²²)₂

The presence of certain substituents in the compounds of formula (1) may enable salts of the compounds to be formed. Suitable salts include pharmaceutically acceptable salts, for example acid addition salts derived from inorganic or organic acids, and salts derived from inorganic and organic bases.

Acid addition salts include hydrochlorides, hydrobromides, hydroiodides, alkylsulphonates, e.g. methanesulphonates, ethanesulphonates, or isothionates, arylsulphonates, e.g. p-toluenesulphonates, besylates or napsylates, phosphates, sulphates, hydrogen sulphates, acetates, trifluoroacetates, propionates, citrates, maleates, fumarates, malonates, succinates, lactates, oxalates, tartrates and benzoates.

Salts derived from inorganic or organic bases include alkali metal salts such as sodium or potassium salts, alkaline earth metal salts such as magnesium or calcium salts, and organic amine salts such as morpholine, piperidine, dimethylamine or diethylamine salts.

Particularly useful salts of compounds according to the invention include pharmaceutically acceptable salts, especially acid addition pharmaceutically acceptable salts.

A particularly useful group of compounds according to the invention has the formula (1a):

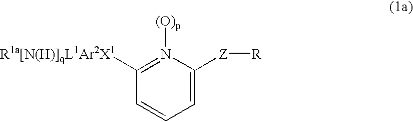

in which R^{1a}, q, L¹, Ar², X¹, p, Z and R are as defined for formula (1); and the salts, solvates, hydrates and N-oxides thereof.

In compounds of formula (1a) and in general in compounds of the invention L¹ is preferably a covalent bond or a —C(O)—, —C(S)— or —[C(R³)(R⁴)]ₙ— group where R³ and R⁴ are as previously generally and particularly defined and n is the integer 1.

In compounds of formula (1a) and in general in compounds of the invention the pyridine or pyridine N-oxide represented by Ar¹ may be optionally substituted with one or two halogen atoms, or one or two straight or branched alkyl, haloalkyl, alkoxy, haloalkoxy, alkylthio groups, —OH, —CO₂H, —CO₂R⁸ e.g. —CO₂CH₃, —CN, —NO₂, —NH₂ or straight or branched alkylamino e.g. —NHCH₃ or dialkylamino e.g. —N(CH₃)₂ groups. Particularly useful groups represented by Ar¹ are unsubstituted pyridine and pyridine N-oxide.

In compounds of formula (1a) and in general in compounds of the invention the group Z is preferably a —CH(R¹³)CH₂— or —C(R¹³)═CH— group. In these compounds the group R¹³ is preferably the group R^{13a} where R^{13a} is an optionally substituted aromatic or heteroaromatic group as herein defined. Particularly useful aromatic groups include optionally substituted phenyl groups. Particularly useful heteroaromatic groups include optionally substituted five- or six-membered heteroaromatic groups, e.g. optionally substituted pyridyl, pyrimidinyl and pyridine-N-oxide groups. A most especially preferred heteroaromatic group is an optionally substituted pyridyl group.

In compounds of formula (1a) and in general in compounds of the invention the optional substituents on R¹³ groups include one or more substituents which may be the same or different selected from halogen atoms, especially fluorine, chlorine or bromine atoms, C₁₋₆alkyl groups, especially methyl, ethyl or i-propyl groups, carboxyl (—CO₂H) or esterified carboxyl (—CO₂Alk⁴) groups especially —CO₂CH₃, amino (—NH₂) or substituted amino groups especially —NHCH₃ and —N(CH₃)₂ groups, aminoC₁₋₆alkylaminocarbonyl groups especially aminoethylaminocarbonyl groups, hydroxyl or C₁₋₆alkoxy groups, especially methoxy, ethoxy or i-propoxy groups, haloC₁₋₆alkoxy groups, especially trifluoromethoxy, thio (—SH) or thioC₁₋₆alkyl groups, especially thiomethyl, nitro, cyano, amidino, C₁₋₆alkylenedioxy groups especially methylenedioxy, C₁₋₆alkylsulphuryl, especially methylsulphuryl or C₁₋₆alkylsulphonyl, especially methylsulphonyl groups.

In compounds of formula (1a) and in general in compounds of the invention, the group R is preferably a carboxylic acid (—CO₂H).

The atom or group X¹ in general and in compounds of formula (1a) is preferably a —O— or —S— atom or —C(O)—, —N(R⁵)—, —C(═NOH), —C(═CR⁵R⁶)— or —C(R⁵)(R⁶)— group. Particularly useful —N(R⁵)— groups include —NH— and —N(CH₃)— groups. Particularly useful —C(R⁵)(R⁶)— groups include those where R⁵ is a hydrogen atom and R⁶ is a hydrogen atom or hydroxyl or alkoxy, especially methoxy, group. Particularly useful —C(═CR⁵R⁶)— groups include —C(═CH₂)— and —C(═C(CH₃)₂) groups. Most preferably X¹ is an —O— atom or a —NH— group.

The group Ar² in general and in compounds of formula (1a) is preferably an optionally substituted 1,4-phenyl, 2,5-pyridyl or 2,5 pyrimidinyl group.

In compounds of formula (1a) and in general in compounds of the invention R^{1a} may in particular be a group R¹⁴R¹⁵NC(X²)— in which X² is preferably a ═NR¹⁶ group, R¹⁵C(═NR¹⁴)—, an optionally substituted five to ten membered, particularly five to seven membered, nitrogen containing mono-or bicyclic fused ring heterocycloaliphatic, optionally containing one or more heteroatoms as described in relation to R^{1a} or an optionally substituted five- to ten membered, particularly five- or six-membered nitrogen containing monocyclic heteroaromatic group or nine- or ten-membered nitrogen containing bicyclic heteroaromatic group, optionally containing one or more other heteroatoms as described in relation to R^{1a}. Particularly useful R^{1a} groups include H₂NC(═NH)— optionally substituted imidazolinyl, imidazolyl, pyridyl, benzimidazolyl, tetrahydropyrimidinyl, tetrahydro-[1,8]-naphthyridinyl, [1,8]-naphthyridinyl and triazolyl groups. Especially useful optionally substituted pyridyl groups include pyridyl, 6-aminopyrid-2-yl and 6-methylaminopyrid-2-yl groups. Especially useful tetrahydropyrimidinyl groups include tetrahydropyrimidin-2-yl groups, especially 1,4,5,6-tetrahydro-5-hydroxy-pyrimidin-2-yl groups. Especially useful tetrahydro-[1,8]-naphthyridinyl groups include 5,6,7,8-tetrahydro-[1,8]-naphthyridin-2-yl groups. Especially useful [1,8]-naphthyridinyl groups include [1,8]-naphthyridin-2-yl groups.

In one preferred class of compounds of formula (1a) q is zero and L¹ is a covalent bond. In this class of compounds R^{1a} is preferably a group as group R¹⁴R¹⁵NC(X²)— in which X² is preferably a ═NR¹⁶ group, or a group R¹⁵C(═NR¹⁴)— or an optionally substituted five- or six-membered, nitrogen containing heterocycloaliphatic or five- to ten-membered nitrogen containing heteroaromatic group, optionally containing one or two other heteroatoms as described herein in relation to R^{1a}. Particularly useful R^{1a} groups in this class of compounds include H₂NC(═NH)— and optionally substituted pyridyl, 5,6,7,8-tetrahydro-[1,8]-naphthyridin-2-yl, [1,8]-naphthyridin-2-yl and imidazolyl groups. Especially useful R^{1a} optionally substituted pyridyl groups include pyrid-2-yl, 6-aminopyrid-2-yl and 6-methylaminopyrid-2-yl groups.

In another preferred class of compounds of formula (1a) q is zero and L¹ is a [C(R³)(R⁴)]ₙ— group in which n is preferably the integer 1 and R³ and R⁴ is each preferably a hydrogen atom or R³ is a hydrogen atom and R⁴ is a hydroxyl (—OH) group. In this class of compounds R^{1a} is preferably a group R¹⁴R¹⁵NC(X²)—, in which X² is preferably ═NR¹⁶,or a group R¹⁵C(═NR¹⁴)— or an optionally substituted five or six-membered nitrogen containing heterocycloaliphatic or five- to ten-membered nitrogen containing heteroaromatic group, optionally containing one or two other heteroatoms as desccibed herein in relation to R^{1a}. Particularly useful R^{1a} groups in this class of compounds include H₂NC(═NH)— and optionally substituted imidazolyl, imidazolinyl, triazolyl, tetrahydro-[1,8]-naphthyridinyl, [1,8]-naphthyridinyl, and pyridyl groups. Especially useful optionally substituted pyridyl groups include pyrid-2-yl, 6-aminopyrid-2-yl and 6-methylaminopyrid-2-yl groups.

In another preferred class of compounds of formula (1a) q is the integer 1 and L¹ is a —C(O)— or —[C(R³)(R⁴)]ₙ— group where n is the integer 1, and R³ and R⁴ is each preferably a hydrogen atom or C₁₋₆alkyl especially methyl group or a hydroxyl group. An especially preferred group of compounds of this class is that where L¹ is a —C(R³)(R⁴)— group in which R³ and R⁴ is each a hydrogen atom. In this class of compounds R^{1a} is preferably a group R¹⁴R¹⁵NC(X²)— in which X² is preferably ═NR¹⁶, or a group R¹⁵C(═NR¹⁴) or an optionally substituted five to ten-membered nitrogen containing heterocycloaliphatic or five- to ten-membered nitrogen containing heteroaromatic group optionally containing one or two other heteroatoms as described herein in relation to R^{1a}. Especially useful heterocycloaliphatic groups include five- and six-membered heterocycloaliphatic groups. Especially useful heteroaromatic groups include five, six-, nine and ten-membered heteroaromatic groups. Particularly useful R^{1a} groups include H₂NC(═NH)— and optionally substituted imidazolinyl, imidazolyl, benzimidazolyl, 1,4,5,6-tetrahydropyrimidin-2-yl, 5,6,7,8-tetrahydro-[1,8]-naphthyridin-2-yl and optionally substituted pyridyl groups. Especially useful optionally pyridyl groups include pyrid-2-yl, 6-aminopyrid-2-yl and 6-methylaminopyrid-2-yl groups.

In another preferred class of compounds of formula (1) q is the integer 1 and L¹ is a covalent bond. In this class of compounds R^{1a} is preferably a group R¹⁴R¹⁵NC(X²)— in which X² is preferably —NR¹⁶ or a group R¹⁵C(═NR¹⁴) or an optionally substituted five- to ten-membered nitrogen containing heterocycloaliphatic or heteroaromatic group optionally containing one or two other heteroatoms as described herein in relation to R^{1a}. Especially useful heterocycloaliphatic groups include five- and six-membered heterocycloaliphatic gropus. Especially useful heteroaromatic groups include five-, six-, nine- and ten-membered heteroaromatic groups. Particularly useful R^{1a} groups include H₂NC(═NH)— and optionally substituted imidazolinyl, imidazolyl, benzimdazolyl, 1,4,5,6-tetrahydropyrimidin-2-yl, 5,6,7,8-tetrahydro-[1,8]-naphthyridin-2-yl, [1,8]-naphthyridinyl and optionally substituted pyridyl groups. Especially useful optionally substituted pyridyl groups include pyrid-2-yl, 6-aminopyrid-2-yl and 6-methylaminopyridin-2-yl groups.

Particularly preferred optional substituents which may be present on R^{1a} heterocycloaliphatic and heteroaromatic groups include halogen atoms, especially chlorine or fluorine atoms, straight or branched alkyl groups, especially methyl, ethyl or i-propyl groups, haloalkyl groups, especially —CF₃, alkoxy groups, especially methoxy, ethoxy or i-propoxy groups, haloalkoxy groups, especially trifluoromethoxy groups, —OH, —CN, —CO₂H—, —CO₂Alk⁴, especially —CO₂CH₃, —NO₂ or straight or branched alkylamino groups especially methylamino or ethylamino or dialkylamino groups, especially dimethylamino or diethylamino group.

Particularly useful compounds of the invention include:

3-(4-Fluorophenyl)-3-(2-{4-[(2-pyridinylamino)methyl]phenoxy}-pyrid-6-yl-N-oxide)propanoic acid trifluoroacetic acid salt;

3-(3-Pyridinyl)-3-(6-{4-[(2-pyridinylamino)methyl]phenoxy}-2-pyridinyl) propanoic acid;

3-(4-Fluorophenyl)-3-(2-{4-[(1H-1,3-benzimadazol-2-ylamino)methyl] phenoxy]-6-pyridyl-N-oxide)propanoic acid trifluoroacetate salt;

3-(4-Benzoic acid)-3-(6-{4-[(1H-benimidazol-2-ylamino)methyl] phenoxy}-1 -oxypyridin-2-yl)propanoic acid;

3-(4-Benzoic acid)-3-{6-[4-(6-aminopyridin-2-yl)phenoxy]-1 -oxypyridin-2-yl}propanoic acid trifluoroacetate salt;

3-(4-Benzoic acid)-3-(6-{4-[(2-pyridylamino)methyl]phenoxy}-1 -oxypyridin-6-yl)propanoic acid trifluoroacetate salt;

and the salts, solvates, hydrates and N-oxides thereof.

Compounds according to the invention are potent and selective inhibitors of αᵥ integrins. The ability of the compounds to act in this way may be simply determined by employing tests such as those described in the Examples hereinafter.

The compounds are of use in modulating cell adhesion and in particular are of use in the prophylaxis and treatment of diseases or disorders involving inappropriate growth or migration of cells. The invention extends to such a use and to the use of the compounds of formula (1) for the manufacture of a medicament for treating such diseases and disorders. Particular diseases include inflammatory diseases, and diseases involving angiogenesis, bone resorption or cellular or matrix over-expansion.

Particular uses to which the compounds of the invention may be put include the treatment or inhibition of tumour growth and metastasis; retinopathy; macular degeneration psoriasis; rheumatoid arthritis; osteoporosis; bone resorption following or due to joint replacement, hypercalcemia of malignancy, Paget's disease, glucocorticoid treatment, immobilisation-induced osteopenia, hyperparathyroidism or peridontal disease; vascular restenosis; atherosclerosis; inflammatory bowel disease; and psoriasis.

For the prophylaxis or treatment of disease the compounds according to the invention may be administered as pharmaceutical compositions, and according to a further aspect of the invention we provide a pharmaceutical composition which comprises a compound of formula (1) together with one or more pharmaceutically acceptable carriers, excipients or diluents.

Pharmaceutical compositions according to the invention may take a form suitable for oral, buccal, parenteral, nasal, topical or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles and preservatives. The preparations may also contain buffer salts, flavouring, colouring and sweetening agents as appropriate.

Preparations for oral administration may be suitably formulated to give controlled release of the active compound.

For buccal administration the compositions may take the form of tablets or lozenges formulated in conventional manner.

The compounds for formula (1) may be formulated for parenteral administration by injection e.g. by bolus injection or infusion. Formulations for injection may be presented in unit dosage form, e.g. in glass ampoule or multi dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (1) may also be formulated as a depot preparation. Such long acting formulations may be administered by implantation or by intramuscular injection.

For nasal administration or administration by inhalation, the compounds for use according to the present invention are conveniently delivered in the form of an aerosol spray presentation for pressurised packs or a nebuliser, with the use of suitable propellant, e.g. dichlorodifluoromethane, trichlorofluoromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

The quantity of a compound of the invention required for the prophylaxis or treatment of a particular condition will vary depending on the compound chosen, and the condition of the patient to be treated. In general, however, daily dosages may range from around 100 ng/kg to 100 mg/kg e.g. around 0.01 mg/kg to 40 mg/kg body weight for oral or buccal administration, from around 10 ng/kg to 50 mg/kg body weight for parenteral administration and around 0.05 mg to around 1000 mg e.g. around 0.5 mg to around 1000 mg for nasal administration or administration by inhalation or insufflation.

The compounds of the invention may be prepared by a number of processes as generally described below and more specifically in the Examples hereinafter. Many of the reactions described are well-known standard synthetic methods which may be applied to a variety of compounds and as such can be used not only to generate compounds of the invention, but also where necessary the intermediates thereto.

In the following process description, the symbols R, Ar, X¹, Ar¹, L¹, Alk⁴, R⁵, R⁶, and Z when used in the formulae depicted are to be understood to represent those groups described above in relation to formula (1) unless otherwise indicated. In the reactions described below, it may be necessary to protect reactive functional groups, for example hydroxy, amino, thio or carboxy groups, where these are desired in the final product, to avoid their unwanted participation in the reactions. Conventional protecting groups may be used in accordance with standard practice [see, for example, Green, T. W. in “Protective Groups in Organic Synthesis”, John Wiley and Sons, (1999) and the examples herein]. In some instances, deprotection may be the final step in the synthesis of a compound of formula (1) and the processes according to the invention described hereinafter are to be understood to extend to such removal of protecting groups.

Thus according to a further aspect of the invention, a compound of formula (1) in which R is a —CO₂H group may be obtained by hydrolysis of an ester of formula (1b):

Ar—X¹—Ar¹—Z—CO₂Alk⁴ (1b)

where Alk⁴ is a group as previously described.

The hydrolysis may be performed using either an acid or a base depending on the nature of Alk⁴, for example an organic acid such as trifluoroacetic acid optionally in an organic solvent such as a halogenated hydrocarbon e.g. dichloromethane, or an inorganic base such as sodium, lithium or potassium hydroxide optionally in an aqueous organic solvent such as an amide e.g. a substituted amide such as dimethylformamide, an ether, e.g. a cyclic ether such as tetrahydrofuran or dioxane or an alcohol, e.g. methanol at around ambient temperature to 60° C. Where desired, mixtures of such solvents may be used.

Esters of formula (1b) in which X¹ is an —O— or —S— atom or —N(R⁵)— group may be prepared by displacement of a leaving atom or group in a compound of formula (2):

LAr¹ZR (2)

[where L is a leaving atom or group], with a reagent ArX¹H [where X¹ is as just defined].

The reaction may be performed at an elevated temperature, for example the reflux temperature, where necessary in the presence of a solvent, for example a substituted amide such as dimethylformamide, or an ether, e.g. a cyclic ether such as tetrahydrofuran, optionally in the presence of a base, for example a hydride such as sodium hydride, a carbonate such as cesium or potassium carbonate or an organic amine such as pyridine.

Particular examples of leaving groups represented by L in compounds of formula (2) include halogen atoms such as a chlorine or bromine atom, and sulphonyloxy groups, for example alkylsulphonyloxy groups such as a methylsulphonyloxy group.

In a further aspect of the invention esters of formula (1 b) in which X¹ is a —O— or —N(R⁵)— group may be prepared by a coupling reaction of a compound of formula (2a):

HX¹Ar¹ZR (2a)

with a boronic acid of formula ArB(OH)₂ in the presence of a catalyst such as a copper catalyst, e.g. copper(II)acetate and an organic base, for example an amine such as triethylamine or pyridine, where necessary in the presence of a solvent, for example an ether e.g. a cyclic ether such as tetrahydrofuran, a halogenated hydrocarbon e.g. dichloromethane, a nitrile e.g. acetonitrile or an aromatic hydrocarbon e.g. toluene, at a temperature from ambient to the reflux temperature.

In a further aspect of the invention esters of formula (1b) in which X¹ is a —N(R⁵)— group may be prepared by coupling an amine of formula ArN(R⁵)H with a halide of formula (2) [where L is a halogen atom such as a bromine or chlorine atom].

The reaction may be carried out in the presence of a metal complex catalyst such as a palladium complex e.g. dichloro[1,1′-bis(diphenylphosphino)ferrocene]palladium(II), in the presence of an organic base, for example an alkoxide such as sodium t-butoxide, in a solvent such as an ether e.g. a cyclic ether such as tetrahydrofuran, at an elevated temperature e.g. the reflux temperature.

In another process according to the invention a compound of formula (1b) in which X¹ is a —C(R⁵)(R⁶)— group and R is a —CO₂Alk⁴ group may be prepared by cross-coupling a halogen of formula (2b):

Hal²Ar¹ZR (2b)

[where Hal² is a halogen atom such as a chlorine, bromine or iodine atom] with an organometallic reagent ArC(R⁵)(R⁶)MHal³ [where M is a metal atom such as a zinc atom, and Hal³ is a halogen atom such as a bromine atom]. The reaction may be carried out as described hereinafter for the preparation of intermediates of formula (3) under palladium catalysed conditions.

Intermediates of formulae (1b) or (2) in which Ar¹ represents a pyridine N-oxide may be prepared by reaction of intermediates of formulae (1b) or (2) in which Ar¹ represents a pyridine, with an oxidising agent, for example a peroxy acid such as m-chloroperoxybenzoic acid, optionally in the presence of t-butyl peroxide in a solvent, for example an alcohol such as tert-butanol, at an elevated temperature e.g. the reflux temperature. Intermediate compounds of formula (2) in which Z is a —CH(R¹³)CH₂— group and R is a —CO₂Alk⁴ group may be prepared by reaction of an intermediate of formula (3):

LAr¹CH₂R¹³ (3)

with an α-haloester HalCH₂CO₂Alk⁴ [where Hal is a halogen atom such as a chlorine, bromine or iodine atom] in the presence of a strong base, e.g. a silazide such as sodium or lithium hexamethyidisilazide in a solvent such as an ether, e.g. a cyclic ether such as tetrahydrofuran at a low temperature, e.g. around −78° C.

In a similar manner intermediates of formula (2a) or (2b) may be prepared from intermediates of formula (3) in which HX¹— is replaced by a L¹— atom or group or Hal² atom.

In a further method of preparation of intermediate compounds of formula (2) in which Z is a —CH(R¹³)CH₂— group and R is a —CO₂Alk⁴ group reaction of an intermediate of formula (4):

LAr¹CH═CHR (4)

may be reacted with an organometallic reagent such as a Grignard reagent of formula R¹³MgHal [where Hal is a halogen atom such as a chlorine, bromine or iodine atom], optionally in the presence of a copper reagent such as copper (I) bromide dimethyl sulphide complex or copper (I) iodide in the presence of a diamine such as N,N,N′,N′-tetramethylenediamine, or with an organocuparate reagent of formula R¹³Cu in a solvent such as an ether e.g. a cyclic ether such as tetrahydrofuran at a low temperature e.g. around −78 to 40° C.

Intermediates of formula (2) may also be prepared by reaction of an intermediate of formula (4) with a boronic acid, R¹³B(OH)₂, in the presence of a catalyst, for example a rhodium catalyst, e.g. Rh(acac)(C₂H₄)₂ optionally in a solvent, for example an ether e.g. 1,4-dioxane or tetrahydrofuran.

Intermediates of formula (3) may be prepared by cross-coupling a halide of formula (5):

LAr¹CH₂Hal¹ (5)

[where Hal¹ is a halogen atom such as a chlorine atom] with an organometallic reagent R¹³MHal², where M is a metal atom such as a zinc atom, and Hal² is a halogen atom such as a bromine atom.

The reaction may be carried out in the presence of a metal cataylst, for example a metal complex catalyst such as a palladium complex, e.g. tetrakis(triphenylphosphine)palladium (O), in a solvent such as an ether, e.g. a cyclic ether such as tetrahydrofuran, at an elevated temperature e.g. the reflux temperature.

Intermediate compounds of formula (4) in which R is a —CO₂Alk⁴ group may be prepared by reaction of an aldehyde of formula (6):

LAr¹CHO (6)

with a phosphonate (Alk⁵O)₂P(O)CH₂CO₂Alk⁴ in the presence of a base.

Suitable bases include organometallic bases, for example an organolithium compound such as n-butyllithium or lithium diisopropylamide, hydrides such as sodium or potassium hydride, alkoxides, such as sodium alkoxides, e.g. sodium methoxide, and cyclic amines, for example 1,8-diazabiacyclo[5.4.0]undec-7-ene.

The reaction may be performed in a suitable solvent, for example a polar aprotic solvent such as an amide, e.g. N,N-dimethylformamide; or a non-polar solvent such as an ether, e.g. a cyclic ether such as tetrahydrofuran or a halogenated hydrocarbon, e.g. dichloromethane. Preferably the reaction is carried out at a low temperature, for example from around −78° C. to around ambient temperature.

Intermediate aldehydes of formula (6) may be obtained from a halide of formula (7):

LAr¹Hal⁴ (7)

[where Hal⁴ is a halogen atom such as a chlorine, bromine or iodine atom] by halogen-metal exchange with a base such as n-butyllithium, followed by reaction with an electrophile such as dimethylformamide in a solvent such as tetrahydrofuran at a low temperature e.g. around −70° C. and subsequent treatment with an acid such as hydrochloric acid at around ambient temperature.

In a further process intermediates of formula (2) may be prepared from a compound of formula (8) [where R^{d} is a C₁₋₆alkyl group]:

LAr¹CH(R¹³)CH(CO₂R^{d})₂ (8)

by hydrolysis of the ester functionality as previously described to give an intermediate of formula (8) [R^{d}═H], followed by decarboxylation at elevated temperature, for example 100 to 170° C., where necessary in the presence of a solvent, for example a sulphoxide such as dimethylsulphoxide.

Intermediates of formula (8) may be prepared by reaction of an intermediate of formula (9):

LAr¹CH═C(CO₂Alk⁴)₂ (9)

with an organometallic reagent R¹³MgHal as previously described for the synthesis of intermediates of formula (2) from intermediates of formula (4).

Intermediates of formula (9) may be prepared by reaction of a malonate, for example dimethylmalonate with an aldehyde of formula (6) optionally in the presence of an acid source, for example an organic acid e.g. acetic acid and/or an organic base for example an amine e.g. triethylamine or piperadine under conditions where water is eliminated from the reaction, for example Dean and Stark conditions, in a solvent, for example an aromatic hydrocarbon e.g. toluene at an elevated temperature e.g. the reflux temperature.

In a further process intermediates of formula (2) in which Z is a —CH(R¹³)CH₂—group may be prepared from a n intermediate of formula (2) in which Z is a —C(R¹³)═CH— group, by hydrogenation, using a metal catalyst, for example palladium on a support such as carbon, in a solvent such as an alcohol e.g. ethanol in the presence of ammonium formate, cyclohexadiene or hydrogen, at a temperature from around ambient to the reflux temperature.

Intrmediates of formula (2) in which Z is a —C(R¹³)═CH— group may be prepared by the methods previously described for the preparation of intermediate of formula (4), starting from a ketone of formula (10):

LAr¹C(O)R¹³ (10)

In another method of preparation of intermediates of formula (2), in which Z is a —C(R¹³)═CH—group, intermediate compounds of formula (4) may be reacted with arylhalides of formula R¹³Hal⁵ (where Hal⁵ is a halogen atom such as an iodine, bromine or chlorine atom) in the presence of a catalyst, for example a palladium catalyst e.g. palladium(II) acetate optionally in the presence of a tertiary phosphine e.g. triphenyl or tritolylphosphine, optionally in the presence of a base, for example an organic amine base e.g. triethylamine in a solvent for example an aromatic hydrocarbon e.g. toluene, an amide e.g. dimethylformamide, or an ether for example a cyclic ether e.g. tetrahydrofuran at a temperature from around ambient to 100° C.

Intermediate compounds of formula (10) may be prepared by oxidation of alcohols of formula (11):

LAr¹CH(OH)R¹³ (11)

with an inorganic oxidising agent, for example manganese dioxide or potassium permanganate optionally is a solvent, for example a ketone e.g. acetone at ambient temperature.

Intermediate compounds of formula (11) may be prepared from aldehydes of formula (6) by reaction with organometalic reagents such as Grigard reagents of formula R¹³MgHal or lithium reagents of formula R¹³Li is a solvent such as an ether e.g. a cyclic ether such as tetrahydrofuran at a low temperature e.g. around −78 to −40° C.

In a further process intermediate compounds of formula (10) may be formed by reaction of an amide, for example a Weinreb amide of formula (13) [where R^{f} is a C₁₋₆alkyl group] with an organometallic reagent as just described.

LAr¹C(O)N(OR^{f})R^{f} (12)

Intermediate ketones of formula (10) may also be obtained from a halide of formula (13):

LAr¹Hal³ (13)

[where Hal³ is a halogen atom such as a chlorine atom] by halogen-metal exchange with a base such as n-butyllithium, followed by reaction with a nitrile R¹³CN, an acid chloride R¹³COCl, an amide R¹³CON(OMe)Me or an ester R¹³CO₂Alk⁴ in a solvent such as tetrahydrofuran at a low temperature e.g. around −70° C. and subsequent treatment with an acid such as hydrochloric acid at around ambient temperature.

Where in the general processes described above intermediates such as ArX¹H, and the halides of formulae (6) and (7) are not available commercially or known in the literature, they may be readily obtained from simpler known compounds by one or more standard synthetic methods such as those described in the general reference texts Rodd's Chemistry of Carbon Compounds, Volumes 1-15 and Supplementals (Elsevier Science Publishers, 1989), Fieser and Fieser's Reagents for Organic Synthesis, Volumes 1-19 (John Wiley and Sons, 1999), Comprehensive Heterocyclic Chemistry, Ed. Katritzky et al, Volumes 1-8, 1984 and Volumes 1-11, 1994 (Pergamon), Comprehensive Organic Functional Group Transformations, Ed. Katritzky et al, Volumes 1-7, 1995 (Pergamon), Comprehensive Organic Synethesis, Ed. Trost and Flemming, Volumes 1-9, (Pergamon, 1991), Encyclopedia of Reagents for Organic Synthesis, Ed. Paquette, Volumes 1-8 (John Wiley and Sons, 1995), Larock's Comprehensive Organic Transformations (VCH Publishers Inc., 1989) and March's Advanced Organic Chemistry (John Wiley and Sons, 1992), employing for example substitution, oxidation, reduction or cleavage reactions. Particular substitution approaches include conventional alkylation, arylation, heteroarylation, acylation, thioacylation, halogenation, sulphonylation, nitration, formylation and coupling procedures. It will be appreciated that these methods may also be used to obtain or modify other intermediates and in particular compounds of formula (1) where appropriate functional groups exist in these compounds. Particular examples of such methods are given in the Examples hereinafter.

Thus, for example, ester groups such as —CO₂Alk⁴ and —CO₂R⁸ in the compound of formula (1) and intermediates thereto may be converted to the corresponding acid [—CO₂H] by acid- or base-catalysed hydrolysis depending on the nature of the groups R⁸ or Alk⁴. Acid- or base-catalysed hydrolysis may be achieved for example by treatment with an organic or inorganic acid, e.g. trifluoroacetic acid in an organic solvent e.g. dichloromethane or a mineral acid such as hydrochloric acid in a solvent such as dioxan or an alkali metal hydroxide, e.g. lithium hydroxide in an aqueous alcohol, e.g. aqueous methanol.

In a further example amides, for example R^{1a}N(R²)COAr²X¹H, may be obtained by reaction of an amine R^{1a}N(R²)H with an acid HX¹Ar²CO₂H in the presence of a condensing agent, for example a diimide such as 1-(3-dimethylaminopropyl)-3-ethylcarbodiimide hydrochloride or N,N′-dicyclohexylcarbodiimide, or a benzotriazole such as [0-(7-azabenzotriazol-1-yl)-1,1,3,3-tetramethyluronium]hexafluorophosphate, advantageously in the presence of a catalyst such as a N-hydroxy compound e.g. a N-hydroxybenzotriazole such as 1-hydroxybenzotriazole. The reaction may be performed in the presence of a base, such as an amine e.g. triethylamine or N-methylmorpholine optionally in the presence of a catalytic amount of 4-dimethylaminopyridine in a solvent such as a halogenated hydrocarbon e.g. dichloromethane, at for example ambient temperature.

In a further example, —OR²⁰ [where R²⁰ represents an alkyl group such as methyl group] in compounds of formula (1) and intermediates thereto may be cleaved to the corresponding alcohol —OH by reaction with boron tribromide in a solvent such as a halogenated hydrocarbon, e.g. dichloromethane at a low temperature, e.g. around −78° C.

Alcohol [—OH] groups may also be obtained by hydrogenation of a corresponding —OCH₂R²⁰ group (where R²⁰ is an aryl group) using a metal catalyst, for example palladium on a support such as carbon in a solvent such as ethanol in the presence of ammonium formate, cyclohexadiene or hydrogen, from around ambient to the reflux temperature. In another example, —OH groups may be generated from the corresponding ester [e.g. —CO₂Alk⁴] or aldehyde [—CHO] by reduction, using for example a complex metal hydride such as lithium aluminium hydride or sodium borohydride in a solvent such as methanol.

In another example, alcohol —OH groups in the compounds may be converted to a corresponding —OR²⁰ group by coupling with a reagent R²⁰OH in a solvent such as tetrahydrofuran in the presence of a phosphine, e.g. triphenylphosphine and an activator such as diethyl-, diisopropyl-, or dimethylazodicarboxylate.

Aminosulphonylamino [—NHSO₂NH₂] groups in the compounds may be obtained, in another example, by reaction of a corresponding amine [—NH₂] with sulphamide in the presence of an organic base such as pyridine at an elevated temperature, e.g. the reflux temperature.

In a further example amine (—NH₂) groups may be alkylated using a reductive alkylation process employing an aldehyde and a reducing agent. Suitable reducing agents include borohydrides for example sodium triacetoxyborohyride or sodium cyanoborohydride. The reduction may be carried out in a solvent such as a halogenated hydrocarbon, e.g. dichloromethane, a ketone such as acetone, or an alcohol, e.g. ethanol, where necessary in the presence of an acid such as acetic acid at around ambient temperature. Alternatively, the amine and aldehyde may be initially reacted in a solvent such as an aromatic hydrocarbon e.g. toluene and then subjected to hydrogenation in the presence of a metal catalyst, for example palladium on a support such as carbon, in a solvent such as an alcohol, e.g. ethanol.

In a further example, amine [—NH₂] groups in compounds of formula (1) and intermediates thereto may be obtained by hydrolysis from a corresponding imide by reaction with hydrazine in a solvent such as an alcohol, e.g. ethanol at ambient temperature.

In another example, a nitro [—NO₂] group may be reduced to an amine [—NH₂], for example by catalytic hydrogenation using for example hydrogen in the presence of a metal catalyst, for example palladium on a support such as carbon in a solvent such as an ether, e.g. tetrahydrofuran or an alcohol e.g. methanol, or by chemical reduction using for example a metal, e.g. tin or iron, in the presence of an acid such as hydrochloric acid.

In a further example amine (—CH₂NH₂) groups in compounds of formula (1) and intermediates thereto may be obatined by reduction of nitriles (—CN), for example by catalytic hydrogenation using for example hydrogen in the presence of a metal catalyst, for example palladium on a support such as carbon, or Raney® nickel, in a solvent such as an ether e.g. a cyclic ether such as tetrahydrofuran or an alcohol e.g. methanol or ethanol, optionally in the presence of ammonia solution at a temperature from ambient to the reflux temperature, or by chemical reduction using for example a metal hydride e.g. lithium aluminium hydride, in a solvent such as an ether e.g. a cyclic ether such as tetrahydrofuran, at a temperature from 0° C. to the reflux temperature.

Aromatic halogen substituents in the compounds may be subjected to halogen-metal exchange with a base, for example a lithium base such as n-butyl or t-butyl lithium, optionally at a low temperature, e.g. around −78° C., in a solvent such as tetrahydrofuran and then quenched with an electrophile to introduce a desired substituent. Thus, for example, a formyl group may be introduced by using dimethylformamide as the electrophile, a thiomethyl group may be introduced by using dimethyidisulphide as the electrophile and an alcohol group may be introduced by using an aldehyde as electrophile.

In another example, sulphur atoms in the compounds, for example when present in a group L¹ may be oxidised to the corresponding sulphoxide or sulphone using an oxidising agent such as a peroxy acid, e.g. 3-chloroperoxybenzoic acid, in an inert solvent such as a halogenated hydrocarbon, e.g. dichloromethane, at around ambient temperature.

Where desired, imidourea groups, for example N(R²)C(═NR¹⁶)NR¹⁴R¹⁵ represented by R^{1a}N(R²) in compounds of the invention or intermediates thereto may be obtained by reaction of a corresponding amine, for example —NHR², with a guanidine containing a leaving group, e.g. LC(═NR¹⁶)NR¹⁴R¹⁵ where L is a leaving group such as a pyrazole group, in a solvent such as acetonitrile at an elevated temperature.

In a further example N-oxides of compounds of formula (1) may in general be prepared for example by oxidation of the corresponding nitrogen base using an oxidising agent such as hydrogen peroxide in the presence of an acid such as acetic acid, at an elevated temperature, for example around 70° C. to 80° C, or alternatively by reaction with a peracid such as peracetic acid or m-chloroperoxybenzoic acid in a solvent,such as a halogenated hydrocarbon e.g. dichloromethane or an alcohol e.g. tert-butanol at a temperature from the ambient temperature to the reflux temperature.

Salts of compounds of formula (1) may be prepared by reaction of a compound of formula (1) with an appropriate acid or base in a suitable solvent or mixture of solvents e.g. an organic solvent such as an ether e.g. diethylether, or an alcohol, e.g. ethanol using conventional procedures.

Where it is desired to obtain a particular enantiomer of a compound of formula (1) this may be produced from a corresponding mixture of enantiomers using any suitable conventional procedure for resolving enantiomers.

Thus for example diastereomeric derivatives, e.g. salts, may be produced by reaction of a mixture of enantiomers of formula (1) e.g. a racemate, and an appropriate chiral compound, e.g. a chiral base. The diastereomers may then be separated by any convenient means, for example by crystallisation and the desired enantiomer recovered, e.g. by treatment with an acid in the instance where the diastereomer is a salt.

In another resolution process a racemate of formula (1) may be separated using chiral High Performance Liquid Chromatography. Alternatively, if desired a particular enantiomer may be obtained by using an appropriate chiral intermediate in one of the processes described above.

Chromatography, recrystalliation and other conventional separation procedures may also be used with intermediates or final products where it is desired to obtain a particular geometric isomer of the invention.

The following Examples illustrate the invention. All temperatures are in ° C. The following abbreviations are used:

| | | | |
|---|---|---|---|
| THF | tetrahydrofuran; | boc | butoxycarbonyl |
| DMF | dimethylformamide; | DMSO | dimethyl sulphoxide; |
| DCM | dichloromethane; | TFA | trifluoroacetic acid |
| MeOH | methanol; | EtOH | ethanol |
| EtOAc | Ethyl acetate. | nBuLi | n-butyllithium |

All NMR's were obtained at 300 MHz.

Intermediate 1

2-Bromo-6-(4-fluorobenzyl)pyridine

To a stirred suspension of zinc (3.6 g, 5.5 mmol) in THF (100 ml) at reflux was added 4-fluorobenzyl bromide (9.45 g, 6.23 ml, 50 mmol) dropwise. The reaction mixture was refluxed for 1 h and then allowed to cool to room temperature. 2,6-Dibromopyridine (11.85 g, 50 mmol) and tetrakis(triphenyl phosphine)palladium(0) (0.5 g, 0.43 mmol) were added as a solution in THF (100 ml) and the reaction mixture heated to reflux for 3 h. Upon cooling the reaction mixture was poured onto saturated sodium bicarbonate solution (250 ml) and extracted twice with DCM (250 ml). The combined organic fractions were dried (MgSO₄), filtered and the solvent removed in vacuo. Purification by flash chromatography (silica; 4:1 hexane/diethyl ether) gave 10.98 g (85%) of the title compound. ¹H NMR (CDCl₃) δ 7.50-7.40 (2H, m), 7.40-7.18 (3H, m), 7.10-6.95 (3H, m), and 4.08 (2H, s).

Intermediate 2

t-Butyl-3-(2-bromo-6-pyridyl)-3-(4-fluorophenyl)propanoate

To a stirred solution of Intermediate 1 (10.98 g, 41.3 mmol) in THF (200 ml) under a nitrogen atmosphere at −78° was added sodium bistrimethylsilylamide (1M solution in THF, 42 ml, 42 mmol) dropwise. The reaction mixture was stirred for 30 min, and then t-butyl bromoacetate (8.17 g, 6.2 ml, 42 mmol) added dropwise as a solution in THF (50 ml). The reaction mixture was allowed to warm to room temperature, and then poured into saturated sodium bicarbonate solution (300 ml) and the product extracted twice with DCM (200 ml). The combined organic fractions were dried (MgSO₄), filtered and the solvent removed in vacuo. Purification by flash chromatography (silica; 4:1 hexane/diethyl ether) gave 12.32 g (77%) of the title compound. H¹ NMR (CDCl₃) δ 7.50-7.40 (1H, m), 7.33-7.25 (3H, m), 7.12-6.95 (3H, m), 4.53 (1H, t, J 8.0 Hz), 3.28 (1H, dd, J 16.0, 8.0 Hz), 2.88 (1H, dd, J 16.0, 8.0 Hz) and 1.34 (9H, s).

Intemediate 3

t-Butyl-3-(2-bromo-6-pyridyl-N-oxide)-3-(4-fluorophenyl)propanoate

To a stirred solution of Intermediate 2 (1 g, 2.63 mmol) in t-butanol (50 ml) was added excess m-chloroperoxybenzoic acid and t-butyl peroxide. The reaction mixture was heated to reflux for 2 h. Upon cooling the reaction mixture was poured cautiously into saturated sodium metabisulphite solution (100 ml). The product was extracted twice with DCM (50 ml). The combined organic fractions were washed with saturated sodium bicarbonate solution (100 ml) then dried (MgSO₄), filtered and the solvent removed in vacuo. Purification by flash chromatography (silica; diethyl ether) gave 0.56 g (54%) of the title compound. ¹H NMR (CDCl₃) δ 7.57 (1H, dd, J 2.8 Hz), 7.32-7.22 (2H, m), 7.15-6.95 (4H, m), 5.25 (1H, dd, J 8.0, 3.0 Hz), 3.23 (1H, dd, J 16.0, 7.0 Hz), 2.84 (1H, dd, J 16.0, 9.0 Hz) and 1.29 (9H, s).

Intermediate 4

4-[(2-Pyredinylamino)methyl]phenol

4-Hydroxybenzaldehyde (3.9 g, 32 mmol) and 2-aminopyridine (3.0 g, 32 mmol) were stirred in toluene (100 ml) at room temperature for 5 min. After concentrating in vacuo the residue was dissolved in EtOH (50 ml) and hydrogenated over 10% Pd on carbon (100 mg) under a balloon of hydrogen, for 18 h. The reaction mixture was filtered, concentrated and the crude product was purified by chomatography (silica; DCM) to yield the title compound as white crystals (3.6 g, 56%). ¹H NMR δ (CDCl₃) 8.10 (1H, m), 7.49 (1H, m), 7.14 (2H, d, J 7.8 Hz), 6.79 (2H, d, J 6.7 Hz), 6.64 (1H, m), 6.47 (1H, d, J 7.8 HZ), 4.79 (1H, br s) and 4.31 (2H, d, J 5.2 Hz).

Intermediate 5

N-Methoxy-N-methylpyridine-3-carboxamide

Nicotinoyl chloride hydrochloride (4.0 g, 22.5 mmol) and N—O-dimethylhydroxylamine hydrochloride (2.6 g, 27 mmol) was suspended in DCM (30 ml) and treated with triethylamine (8.0 g, 78.8 mmol). The reaction was stirred for 2 h at room temperature then partitioned between DCM and sodium hydrogen carbonate solution. The organic phase was separated, dried (MgSO₄) and concentrated in vacuo to give 3.4 g (91%) of the title compound. ¹H NMR δ (CDCl₃) 8.95 (1H, d, J 1.0 Hz), 8.68 (1H, d, J 4.3 Hz), 7.99 (1H, m), 7.32 (1H, m), 3.58 (3H, s), 3.39 (3H, s).

Intermediate 6

(6-Bromo-2-pyridinyl)-3-pyridinylmethanone

2,6-Dibromopyridine (4.30 g, 18.1 mmol) in THF (25 ml) was added to nBuLi (11.3 ml, 18.1 mmol, 1.6M in THF) under nitrogen at −78°. After the addition was complete the reaction was stirred at −78° for 15 min and then Intermediate 5 (3.00 g, 18.1 mmol) in THF (5 ml) was added. After stirring at −78° for 15 min the reaction was quenched with 10% aqueous HCl, extracted into diethyl ether, dried and concentrated in vacuo. Chromatography (SiO₂; diethyl ether) yielded 3.97 g (84%) of the title compound. ¹H NMR (d⁶-DMSO) δ 9.12 (1H, s), 8.92 (1H, d, J 4.3 Hz), 8.33 (1H, d, J 8.2 Hz), 8.14-7.94 (3H, m), 7.61 (1H, dd, J 7.0, 5.2 Hz).

Intermediate 7

3-Pyridinyl-(6-{4-[(2-pyridinylamino)methyl]phenoxy}-2-pyridinyl)methanone

Intermediate 4 (2.18 g, 10.9 mmol) and Intermediate 6 (2.87 g, 10.9 mmol) in dioxane (25 ml) were treated with NaH (482 mg, 12 mmol) and heated under reflux for 24 h. The reaction was treated with water and extracted into DCM, dried (MgSO₄) and concentrated in vacuo. Chromatography (SiO₂; diethyl ether-EtOAc, gradient elution) yielded 2.53 g (63%) of the title compound. ¹H NMR (CDCl₃) δ 9.07 (1H, s), 8.73 (1H, m), 8.29 (1H, d, J 7.8 Hz), 8.17 (1H, m), 7.92 (2H, m), 7.50-7.38 (3H, m), 7.29-7.19 (2H, m), 7.12 (2H, d, J 7.8 Hz), 6.60 (1H, dd, J 7.0, 5.2 Hz), 6.49 (1H, d, J 9.5 Hz), 5.91 (1H, br s), 4.57 (2H, d, J 6.1 Hz).

Intermediate 8

t-Butyl-3-[2-(4-cyanoanilino)-6-pyridyl]-3-(4-fluorophenyl)propanoate

To a mixture of Intermediate 2 (1.9 g, 5.0 mmol) 4-cyanoaniline (0.89 g, 7.5 mmol) and dichloro[1,1′-bis(diphenylphosphino)ferrocene]palladium (II) (0.18 g, 0.25 mmol) was added DMF (10 ml). Sodium t-butoxide (0.72 g, 7.5 mmol) was added in a solution in DMF (10 ml). The reaction mixture was heated to 80° C. for 2 h. Upon cooling to room temperature the reaction mixture was poured into saturated sodium hydrogen carbonate solution and extracted twice with DCM. The combined organic fractions were dried over magnesium sulphate, filtered and the solvent removed by evaporation in vacuo. Chromatography (silica; diisopropylether) gave the title compound (0.55 g, 26%). ¹H NMR (CDCl₃) δ 7.50-7.40 (3H, m), 7.30-7.20 (2H, m), 7.00-6.90 (2H, m), 6.70-6.55 (4H, m), 4.45 (1H, t, J 8.0 Hz), 4.10 (1H, br s), 3.25 (1H, dd, J 16.0, 8.0 Hz), 2.88 (1H, dd, J 16.0, 8.0 Hz) and 1.30 (9H, s). m/z (ES⁺, 70V) 418 (MH⁺).

Intermediate 9

t-Butyl-3-[2-[4-(aminomethyl)anilino]-6-pyridyl]-3-(4-fluorophenyl) propanoate

To washed Raney nickel (water ×3, EtOH ×4 (1 g)) in EtOH (20 ml) was added Intermediate 8 (0.55 g, 1.32 mmol) as a solution in EtOH (5 ml). Concentrated ammonia solution (1 ml) was added. The reaction mixture was placed under a hydrogen atmosphere and stirred for 24 h. The reaction mixture was filtered and the solvent removed by evaporation in vacuo. The title compound (0.5 g, 91%) was used crude. ¹H NMR (CDCl₃) δ 8.83 (1H, br s), 7.61-7.50 (2H, m), 7.40, 7.20 (3H, m), 7.1-7.0 (2H, m), 7.0-6.4 (1H, m), 6.70-6.5 (3H, m), 44 (1H, t, J 8.0 Hz), 3.82 (1H, s), 3.25 (1H, dd, J 16.0, 8.0 Hz), 2.87 (1H, dd, J 16.0, 8.0 Hz), 1.3 (9H, s). m/z (ES⁺, 70V) 422 (MH⁺).

Intermediate 10

t-Butyl-3-(2-{[(N,N′-bis-boc([amino]imino)methyl)amino]methyl}anilino)-6-pyridyl)-3-(4-fluorophenyl)propanoate

Intermediate 9 (0.5 g, 1.2 mmol), N,N′-bis-boc-guanyltriflate (0.47 g, 1.2 mmol) and triethylamine (0.12 g, 1.2 mmol) were stirred in DCM (10 ml) for 12 h. The solvent was removed by evaporation in vacuo. Chromatography (silica; diisoproyl ether) gave the title compound (0.42 g, 53%) as a white foam. ¹H NMR (CDCl₃) δ 8.55 (1H, br s), 7.40-7.28 (7H, m), 7.00-6.90 (2H, m), 6.62-6.55 (2H, m), 4.58 (2H, br d, J 5.0 Hz), 4.42 (1H, t, J 8.0 Hz), 3.32 (1H, dd, J 16.0, 8.0 Hz), 2.88 (1H, dd, J 16.0, 8.0 Hz), 1.52 (9H, s), 1.43 (9H, s) and 1.30 (9H, s). m/z (ES⁺, 70V) 663 (MH⁺).

Intermediate 11

4-Benzyloxybenzonitrile

To a stirred solution of 4-cyanophenol (50 g, 0.42 mol) and potassium carbonate (150 g, 1.1 mol) in DMF (800 ml) was added benzyl bromide (75 ml, 0.63 mol). The reaction mixture was stirred for 2 h at room temperature before filtering off solid and reducing the filtrate in vacuo to give an oil. The solid precipitate was dissolved in water and the pH adjusted to 0.5 using 6.0M hydrochloric acid and extacted into EtOAc. The solvent was dried (MgSO₄) and removed by evaporation in vacuo to give an oil. The two oil products were combined and triturated with diethyl ether/hexane to give a white solid, which was washed with hexane and dried to give the title compound (81 g, 93%). ¹H NMR (CDCl₃) δ 7.50 (2H, d, J 8.6 Hz), 7.45-7.30 (5H, br m), 7.00 (2H, d, J 8.6 Hz) and 5.14 (2H, s). m/z (ES⁺, 70V) 210 [M+H]⁺.

Intermediate 12

4-Benzyloxybenzylamine

To a stirred suspension of lithium alminium hydride (1.75 g, 0.46 mol) in THF (800 ml) at 0° was added Intermediate 11 (43.0 g, 0.23 mol) in THF (600 ml), dropwise over 4 h. The reaction mixture was allowed to warm to room temperature and stirred for 16 h and then cooled to 0°. Water (30 ml) was added and 2M sodium hydroxide solution (80 ml) was then added dropwise with stirring. The resulting precipitate was filtered off washed with diethyl ether (100 ml) and toluene (200 ml). The fitrate was washed with sodium chloride solution, dried over sodium sulphate and the solvent removed by evaporation in vacuo, to give a waxy solid. The two solids were combined to give the title compound (48.26 g, 110%). ¹H NMR (CDCl₃) δ 7.46-7.25 (5H, br m), 7.23 (2H, d, J 8.7 Hz), 6.95 (2H, d, J 8.7 Hz), 5.07 (2H, s), 3.81 (2H, s) and 1.50 (2H, br s). m/z; (ES⁺, 70V) 197 [M+NH₄]⁺.

Intermediate 13

1-(2-Aminophenyl)-3-(4-benzyloxybenzyl)-2-thiourea

To a stirred solution of 1,1′-thiocarbonyldiimidazole (12.5 g, 70 mmol) and imidazole (0.95 g, 140 mmol) in acetonitrile (250 ml) at 0° was added Intermediate 12 (11.46 g, 53.8 mmol) in acetonitrile (150 ml) dropwise. The reaction mixture was allowed to warm to room temperature and stirred for 2.5 h and then 1,2-phenylene diamine (10.2 g, 90 mmol) was added. The reaction mixture was stirred overnight. The cream precipitate was filtered off and dried to give the title compound (14.8 g, 88%). ¹H NMR (d⁶ DMSO) δ 8.87 (1H, s), 7.63 (1H, br s), 7.45-7.28 (5H, b m), 7.25 (2H, d J 8.7 Hz), 7.00-6.93 (4H, m), 6.74 (1H, dd, J 8.3, 1.3 Hz), 6.56 (1H, td, J 7.5, 1.4 Hz), 5.08 (2H, s), 4.62 (2H, d, J 5.4 Hz) and 3.32 (2H, s). m/z (ES⁺, 70V) 364 [M+H]⁺.

Intermediate 14

2-(4-{Benzyloxybenzyl}amino)benzimidazole

A stirred solution of Intermediate 13 (3.63 g, 0.01 mol), mercuric oxide (4.33 g, 0.02 mol) and sulphur (64 mg, 0.03 mol) in ethanol (100 ml) was heated under reflux for 48 h. Upon cooling the reaction mixture was filtered through Celite® and the filtrate solvent removed by evaporation in vacuo. The crude product was purified by flash column chromatography (silica; ethyl acetate-90% ethyl acetate/10% methanol) to give the title compound as a white solid (1.50 g, 46%). ¹H NMR δ 10.72 (1H, br s), 7.44-7.39 (5H, br m), 7.29 (2H, d, J 8.6 Hz), 7.11 (1H, v br s), 7.10 (1H, d, J 8.9 Hz), 6.95 (2H, d, J 8.7 Hz), 6.99-6.90 (1H, br m), 6.84 (2H, dd, J 5.9, 3.7 Hz), 5.07 (2H, s) and 4.42 (2H, d, J 5.9 Hz). m/z (ES⁺, 70V) 330 [M+H]⁺.

Intermediate 15

2-(4-{Hydroxybenzyl}amino)benzimidazole

Intermediate 14 (10.07 g, 0.03 mmol) in ethanol (200 ml) was hydrogenated at atmospheric pressure over 10% palladium on carbon (200 mg). The catalyst was removed by filtration and the filtrate concentrated in vacuo yielding the title compound (7.30 g, 100%). ¹H NMR (d⁶ DMSO) δ 10.68 (1H, br s), 9.24 (1H, br s), 7.19-7.10 (5H, m), 6.90-6.81 (4H, m), 6.70 (2H, d, J 8.5 Hz) and 4.37 (2H, d, J 5.6 Hz). m/z (ES⁺, 70V) 240 [M+H]⁺.

Intermediate 16

4-[(6-Bromo-2-pyridinyl)(hydroxy)methyl]benzonitrile

2,6-Dibromopyridine (11.85, 50 mmol) was added to a solution of iso-propylmagnesium bromide (2M in Et₂O, 25 ml, 50 mmol) in tetrahydrofuran (25 ml) at room temperature. The reaction was heated at reflux for 1 h, then treated with 4-cyanobenzaldehyde (6.56 g, 50 mmol). The reaction was heated at reflux for a further 2 h, quenched with water and extracted into DCM. The organic phase was separated, dried over sodium sulphate and concentrated in vacuo. Purification by flash chromatography (silica; DCM) gave the title compound (9.8 g). ¹H NMR (CDCl₃) δ 7.64 (2H, d, J 8.3 Hz), 7.52 (3H, m), 7.42 (1H, d, J 7.8 Hz), 7.15 (1H, d, J 7.5 Hz), 5.80 (1H, s) and 4.47 (1H, s).

Intermediate 17

4-[(6-Bromo-2-pyridinyl){[t-butyl(dimethyl)silyl]oxy}methyl]benzonitrile

2,4,6-Collidine (8.99 ml, 68.0 mmol) was added in a solution of Intermediate 16 (9.8 g, 34.0 mmol) in DCM (50 ml) at 0°. After 5 min t-butyidimethyl-silyltriflate (8.60 ml, 37.4 mmol) was added and the reaction stirred overnight whilst warming to room-temperature. After diluting with DCM the reaction mixture was washed with 1M HCl, water, brine, then the organic phase dried (Na₂SO₄) and concentrated in vacuo. Purification by flash chromatography (silica; hexane:EtOAc 9:1) gave the title compound (12.2 g). ¹H NMR (CDCl₃) δ 7.64-7.57 (4H, m), 7.55-7.47 (2H, m), 7.32 (1H, dd, J 7.0, 1.7 Hz), 5.87 (1H, s), 0.93 (9H, s) and 0.02 (6H, d, J 3.2 Hz). m/z (ES⁺, 70V) 405 (MH⁺).

Intermediate 18

Ethyl-3-{(6-[(t-butyl(dimethyl)silyl)oxy](4-cyanophenyl)methyl)-2-pyridinyl}propanoate

[(Ethoxycyclopropyl)oxy]trimethylsilane (2.36 ml, 13.1 mmol) was added to refluxing zinc chloride (1.79 g, 13.1 mmol) in tetrahydrofuran (15 ml) After 1 h, the reaction was cooled and Intermediate 17 (4.8 g, 11.9 mmol) and tetrakis(triphenylphosphine) palladium (O) (416 mg, 0.36 mmol) were added, and the reaction heated under reflux for 1 h, cooled, quenched with water, extracted into DCM, dried (Na₂SO₄) and concentrated in vacuo. Purification by flash chromatography (silica; heptane: EtOAc 6:1) gave the title compound (4.0 g). ¹H NMR (CDCl₃) δ 7.64-7.52 (5H, m), 7.30 (1H, d, J 7.7 HZz, 7.02 (1H, d, J 7.6 Hz), 5.86 (1H, s), 4.10 (2H, q, J 7.1 Hz), 3.08 (2H, m), 2.77 (2H, t, J 7.4 Hz), 1.24 (3H, t, J 7.1 Hz), 0.93 (9H, s) and 0.01 (6H, d, J 13.6 Hz). m/z (ES⁺, 70V) 425 (MH⁺).

Intermediate 19

Ethyl 3-{(6-[(4-(aminomethyl)phenyl]{[t-butyl(dimethyl)silyl]oxy}methyl)-2-pyridinyl}propanoate

Intermediate 18 (518 mg, 1.2 mmol) was added to a suspension of Raney Nickel (˜100 mg) in ethanol (100 ml) and treated with aqueous ammonia (˜2 ml). This reaction mixture was hydrogenated under a hydrogen atmosphere for 1.5 h. DCM was added and the solution filtered through Celite® and evaporate in vacuo to give the title compound (400 mg). ¹H NMR (CDCl₃) δ 7.50 (1H, t, J 7.7 Hz), 7.43 (2H, d, J 8.0 Hz), 7.32 (1H, d, J 7.7 Hz), 7.20 (2H, d, J 8.2 Hz), 6.96 (1H, d, J 7.4 Hz), 5.82 (1H, s), 4.09 (2H, q, J 7.1 Hz), 3.79 (2H, s), 3.06 (2H, m), 2.74 (2H, m), 2.00 (2H, s), 1.19 (3H, t, J 7.0 Hz), 0.92 (9H, s) and −0.02 (6H, d, J 14.9 Hz). m/z (ES⁺, 70V) 429 (MH⁺).

Intermediate 20

Ethyl-3-{(6-[(t-butyl(dimethyl)silyl)oxy](4-[(2-pyridinyl amino)methyl]phenyl)-2-pyridinyl}propanoate

Intermediate 19 (3.8 g, 8.9 mmol) and 2-fluoropyridine (2.06 ml, 35.5 mmol) were heated together at 100° for 24 h then 120° for a further 24 h. Column chromatography (silica; heptane:EtOAc 3:7) yielded the title compound (1.14 g) together with the compound of Example 13 (0.4 g). ¹H NMR (CDCl₃) δ 8.09 (1H, d, J 3.5 Hz), 7.53 (1H, t, J 7.7 Hz), 7.44 (2H, d, J 8.1 Hz), 7.40 (1H, m), 7.32 (1H, d, J 7.7 Hz), 7.26 (2H, m), 6.98 (1H, d, J 7.5 Hz), 6.57 (1H, dd, J 6.95, 5.2 Hz), 6.36 (1H, d, J 8.4 Hz), 5.83 (1H, s), 4.82 (1H, br t), 4.45 (2H, d, J 5.5 Hz), 4.10 (2H, q, J 7.2 Hz), 3.07 (2H, m), 2.77 (2H, t, J 7.4 Hz), 1.21 (3H, t, J 7.1 Hz), 0.92 (9H, s) and 0.0 (6H, d, J 16.0 Hz). m/z (ES⁺, 70V) 506 (MH⁺).

Intermediate 21

Methyl 4-(6-bromopyrid-2-ylmethyl)benzoate

The methyl-4-(bromomethyl)benzoate (9.7 g, 46.4 mmol) was added to a suspension of zinc (3.04 g, 46.4 mmol) in refluxing THF (8 ml). After 1 h the reaction was cooled and 2,6-dibromopyridine (10 g, 42.2 mmol) and tetrakis(triphenylphosphine) palladium (0) (0.40 g) was added. The reaction was refluxed for 2 h, cooled and quenched with sodium bicarbonate solution sat. The product was extracted with diethyl ether (x 3), dried (MgSO₄) and evaporated in vacuo. Chromatograph (silica; 7:3 hexane:diethyl ether) gave the title compound (6.78 g).

Intermediate 22

Methyl 4-[1-(6-bromo-pyrid-2-yl)-2-methoxycarbonylethyl]-benzoate

To a solution of Intermediate 21 (6.78 g, 22.2 mmol) in THF (40 ml) cooled to −78° was added sodium hexamethyldisilylazide (24.4 ml, 24.4 mmol). After 15 min methyl bromoacetate (2.31 ml, 24.4 mmol) was added and the reaction was left to stir for 1 h, quenched with sodium bicarbonate solution and extracted into DCM (×3), dried (MgSO₄) and evaporated in vacuo. Purification by column chromatography (silica; ether:hexane 7:1) gave the title compound as a colourless oil (7.3 g). ¹H NMR (CDCl₃) δ 7.96 (2H, d, J 8.0 Hz), 7.41-7.35 (3H, m), 7.29-7.27 (1H, d, J 8.0 Hz), 7.00 (1H, d, J 8.0 Hz), 4.66-4.63 (1H, t, J 7.5 Hz), 3.86 (3H, s), 3.59 (3H, s), 3.45-3.37 (1H, dd, J 17.0, 8.0 Hz) and 2.99-2.92 (1H, dd, J 17.0, 7.0 Hz). m/z (ES⁺, 70V) 378 (MH⁺).

Intermediate 23

Methyl 4-[1-(6-bromo-1-oxy-pyridin-2-yl)-2-methoxycarbonylethyl]benzoate

Trifluoroacetic acid (1.2 ml) and hydrogen peroxide (1.2 ml) were added to Intermediate 22 (570 mg, 1.51 mmol) and the mixture was heated at 105° for 4 h. Sodium sulphite solution was added, and the product was extracted into DCM. NMR showed acid/ester mixture which was re-esterified. The product was dissolved in methanol (50 ml) and trimethylsilyl chloride (18 ml) was added to the mixture which was stirred overnight, then evaporated in vacuo. Purification by column chromatography (silica; 5% methanol/DCM) gave the title compound. ¹H NMR (CDCl₃) δ 7.96 (2H, d, J 8.0 Hz), 7.58-7.55 (1H, dd, J 8.0, 2.0 Hz), 7.37 (2H, d, J 8.0 Hz), 7.12 (1H, d, J 2.0 Hz), 7.03 (1H, d, J 8.0 Hz), 5.33-5.25 (1H, m), 3.87 (3H, s), 3.56 (3H, s), 3.33-3.25 (1H, dd, J 16.0, 7.0 Hz), 3.05-3.01 (1H, dd, J 16.0, 7.0 Hz). m/z (ES⁺, 70V) 394 (MH⁺).

Intermediate 24

2-(4-Benzyloxyphenyl)-6-chloropyridine

1-Benzyloxy-4-bromobenzene (6.0 g, 22.8 mmol) was dissolved in THF and cooled to −78° under an inert atmosphere. nBuLi 1.6M (15.7 ml, 25.1 mmol) was added and the reaction stirred for 0.5 h. Zinc chloride (3.42 g, 25.1 mmol) was added and the reaction stirred for a further 1 h. The mixture was warmed to room temperature and 2,6-dichloropyridine (4.05 g, 27.4 mmol) and tetrakis(triphenylphosphine) palladium (0) (0.79 mg, 0.68 mmol) and the reaction was heated to reflux for 18 h. The solution was cooled and the product extracted between DCM and water, dried (MgSO₄) and evaporated in vacuo to leave a cream solid. The product was purified by recrystallisation from heptane to give the title compound as a white solid (7 g). ¹H NMR (CDCl₃) δ 7.96 (2H, d, J 8.9 Hz), 7.66 (1H, t, J 7.7 Hz), 7.57 (1H, d, J 7.7 Hz), 7.57-7.33 (5H, m), 7.19 (1H, d, J 7.7 Hz), 7.06 (2H, d, J 8.9 Hz) and 3.13 (2H, s).

Intermediate 25

[6-(4-Benzyloxyphenyl)pyridin-2-yl]-(4-methoxybenzyl)amine

Intermediate 24 (4 g, 13.5 mmol), 4-methoxybenzylamine (2.75 ml, 20.25 mmol), sodium t-butoxide (2.04 g, 20.25 mmol) and dichloro[1,1′bis-(diphenylphosphine)ferrocene palladium(II) DCM (620 mg) were stirred in THF (30 ml) overnight. Purification was by column chromatography (silica; 4:1 hexane:EtOAc) gave the title product (2.5 g). ¹H NMR (CDCl₃) δ 7.98 (2H, d, J 8.7 Hz), 7.49-7.37 (6H, m), 7.34 (2H, d, J 8.6 Hz), 7.06 (2H, d, J 8.7 Hz), 7.02 (1H, d, J 7.6 Hz), 6.90 (2H, d, J 8.6 Hz), 6.29 (1H, d, J 8.2 Hz), 5.13 (2H, s), 4.96 (1H, t, J 5.3 Hz), 4.52 (2H, d, J 5.6 Hz), 3.81 (3H, s). m/z (ES⁺, 70V) 397 (MH⁺).

Intermediate 26

4-[6-(4-Methoxybenzylamino)pyridin-2-yl]phenol

Intermediate 25 (10 g, 25.25 mmol) and palladium on charcoal (10%) (100 mg) was dissolved in EtOAc (25 ml) under a hydrogen atmosphere. The reaction was stirred overnight. The mixture was filtered through Celite® and evaporated in vacuo down to give the title compound as a brown oil (0.75 g). ¹H NMR (CDCl₃) δ 7.73 (2H, d, J 8.6 Hz), 7.45 (1H, t, J 7.9 Hz), 7.26 (2H, d, J 8.6 Hz), 6.93 (1H, d, J 7.5 Hz), 6.85 (2H, d, J 8.6 Hz), 6.73 (2H, d, J 8.6 Hz). m/z (ES⁺, 70V) 307 (MH⁺).

### EXAMPLE 1

t-Butyl-3-(4-fluorophenyl)-3-(2-{4-[(2-pyridinylamino)methyl}phenoxy]-pyrid-6-yl-N-oxide)propanoate

To Intermediate 4 (0.49 g, 1.24 mmol) in DMF (5 ml) under a nitrogen atmosphere was added sodium hydride in a single portion (0.052 g, 1.3 mmol, 60% dispersion in oil). The reaction mixture was stirred for 30 min. Intermediate 3 (0.49 g, 1.24 mmol) was added as a solution in THF (20 ml), and the reaction mixture was heated to 100° for 4 h. Upon cooling the reaction mixture was poured into saturated sodium bicarbonate solution (50 ml) and extracted twice with DCM (50 ml). The combined organic fractions were dried (MgSO₄), filtered and the solvent removed by evaporation in vacuo. Purification by flash chromatography (silica; EtOAc), gave 0.25 g (39%) of the title compound ¹H NMR (CDCl₃) δ 8.09 (1H. dd, J 4.0, 1.0 Hz), 7.41-7.30 (5H, m), 7.11 (1H, t, J 8.0 Hz), 7.05-6.90 (5H, m), 6.60-6.55 (1H, m), 6.39 (1H, d, J 9.0 Hz), 5.30 (1H, dd, J 9.0, 6.0 Hz), 5.00 (1H, br s), 4.51 (2H, d, J 6.0 Hz), 3.28 (1H, dd, J 16.0, 7.0 Hz), 2.87 (1H, dd, J 16.0, 9.0 Hz) and 1.29 (9H, s).

### EXAMPLE 2

3-(4-Fluorophenyl)-3-(2-{4-[(2-pyridinylamino)methyl]phenoxy}-pyrid-6-yl-N-oxide)propanoic Acid Trifluoroacetic Acid Salt

The compound of Example 1 (0.4 g, 0.78 mmol) was dissolved in 1:1 DCM TFA acid (20 ml) and the reaction mixture was stirred for 72 h at room temperature. The solvent was removed by evaporation in vacuo. Purification by flash chromatography (silica; 15% EtOH/85% DCM/1% TFA) gave the title compound 0.32 g (89%),¹H NMR (DMSO d⁶) δ 8.98 (1H, br s), 7.95 (1H, d, J 6.0 Hz), 7.89 (1H, t, J 7.0 Hz), 7.45-7.30 (6H, m), 7.18 (1H, dd, J 8.0, 2.0 Hz), 7.12-7.05 (3H, m), 6.93-6.82 (3H, m), 5.08 (1H, t, J 7.0 Hz), 4.53 (2H, s), 3.17 (1H, dd, J 16.0, 8.0 Hz) and 2.99 (1H, dd, J 16.0, 8.0 Hz). m/z (ES⁺, 70V) 460 (MH⁺).

### EXAMPLE 3

t-Butyl-3-(4-fluorophenyl)-3-(2-{4-[(2-pyridinylamino)methyl]phenoxy}-pyrid-6-yl)propanoate

To a stirred solution of the compound of Example 1 (400 mg, 0.76 mmol) in EtOH (20 ml) was added excess cyclohexane (5 ml) followed by 10% palladium on carbon (0.2 g). The reaction mixture was heated to reflux for 6 h. Upon cooling the reaction mixture was filtered and the solvent removed by evaporation in vacuo. Purification by flash chromatography (silica; diethyl ether) gave the title compound 0.13 g (33%). ¹H NMR (CDCl₃) δ 8.12 (1H, dd, J 6.0, 1.0 Hz), 7.55 (1H, t, J 8.0 Hz), 7.45-7.20 (5H, m), 7.08 (2H, d, J 8.0 Hz), 6.95-6.82 (3H, m), 6.65 (1H, d, J 8.0 Hz), 6.59 (2H, d, J 6.0 Hz), 6.41 (1H, d, J 8.0 Hz), 4.98 (1H, br s), 4.53 (2H, d, J 7.0 Hz), 3.10 (1H, dd, J 16.0, 8.0 Hz), 2.73 (1H, dd, J 16.0, 8.0 Hz) and 1.24 (9H, s).

### EXAMPLE 4

3-(4-Fluorophenyl)-3-(2-{4-[(2-pyridylamino)methyl]phenoxy}-6-pyrid-6-yl)propanoic Acid Trifluoroacetic Acid Salt

The title compound (0.1 g, 87%) was prepared from the compound of Example 3 (0.13 g, 0.25 mmol) in a similar manner to the compound of Example 2. ¹H NMR (DMSO d⁶) δ 7.96 (1H, dd, J 5.0,1.0 Hz), 7.69 (1H, t, J 8.0 Hz), 7.40-7.30 (3H, m), 7.30-7.20 (2H, m), 7.10-6.95 (5H, m), 6.72 (1H, d, J 7.0 Hz), 6.50 (1H, d, J 8.0 Hz), 6.51-6.42 (1H, m), 4.49 (2H, br s), 4.40 (1H, t, J 8.0 Hz), 2.98 (1H, dd, J 16.0, 8.0 Hz) and 2.70 (1H, dd, J 16.0, 7.0 Hz). m/z (ES⁺, 70V) 444 (MH⁺).

### EXAMPLE 5

(E)-and (Z) Ethyl-3-(3-pyridinyl)-3-(6-{4-[(2-pyridinylamino) methyl]phenoxy}pyrid-2-yl)propenoic Acid

Intermediate 7 (2.53 g, 6.62 mmol) in THF (20 ml) was treated with NaH (584 mg, 14.6 mmol) and stirred until the reaction subsided. Triethylphosphonoacetate (1.48 g, 1.31 ml, 1.62 mmol) was added dropwise under nitrogen and the reaction stirred for 2 h after the addition was complete. The reaction mixture was quenched with sodium hydrogen carbonate solution (saturated) and the aqueous phase extracted repeatedly with DCM. After drying (MgSO₄) the organic extracts were concentrated in vacuo and purified by radial chromatography (diethyl ether/EtOAc, gradient elution), yielding two fractions—the E and Z isomers (0.95 g and 0.70 g respectively) of the title compound.

E isomer

¹H NMR (CDCl₃) δ 8.63 (1H, dd, J 4.9, 1.7 Hz), 8.43 (1H, dd, J 2.2, 0.7 Hz), 8.15 (1H, br d, J 9.0 Hz), 7.62 (1H, dd, J 8.1, 7.8 Hz), 7.54 (1H, br d, J 7.8 Hz), 7.45 (1H, br t, J 8.7 Hz), 7.42 (2H, d, J 8.5 Hz), 7.33 (1H, dd, J 7.8, 4.9 Hz), 7.14 (2H, d, J 8.5 Hz), 7.00 (1H, s), 6.90 (1H, d, J 8.1 Hz), 6.73 (1H, d, J 7.9 Hz), 6.63 (1H, t, J 6.5 Hz), 6.47 (1H, d, J 8.4 Hz), 5.22 (1H, br s), 4.57 (2H, d, J 5.6 Hz), 4.02 (2H, q, J 7.1 Hz), 1.07 (3H, t, J 7.1 Hz).

Z-isomer

¹H NMR (CDCl₃) δ 8.58 (2H, m), 8.12 (1H, m), 7.75 (1H, dd, J 8.2, 6.4 Hz), 7.62 (1H, m), 7.43 (1H, m), 7.33 (2H, d, J 8.6 Hz), 7.26 (1H, m), 7.11 (2H, d, J 8.6 Hz), 7.02 (1H, d, J 8.0 Hz), 6.87 (1H, d, J 8.2 Hz), 6.62 (1H, dd, J 7.1, 5.1 Hz), 6.42 (2H, m), 5.06 (1H, br s), 4.50 (2H, d, J 5.6 Hz), 4.08 (2H, q, J 7.1 Hz), 1.18 (3H, t, J 7.1 Hz).

### EXAMPLE 6

(E)-3-(3-Pyridinyl)-3-(6-{4-[(2-pyridinylamino)methyl]phenoxy}-2-pyridinyl)propenoic Acid

The compound of Example 5 ((E)-isomer) (0.94 g, 2.08 mmol) in THF (3 ml) was treated with water (3 ml) and lithium hydroxide (100 mg, 4.16 mmol). After stirring for 24 h the tetrahydrofuran was removed in vacuo, the residue diluted with water, washed once with diethyl ether, neutralised with 10% HCl solution and extracted repeatedly into DCM. The extract was dried (MgSO₄), filtered and concentrated in vacuo. Freeze-drying from methanol-water yielded the title compound (0.74 g, 84%). ¹H NMR (d⁶-DMSO) δ 8.59 (1H, d, J 5.2 Hz), 8.41 (1H, s), 8.02 (1H, d, J 5.2 Hz), 7.87 (1H, t, J 7.8 Hz), 7.63 (1H, m), 7.41 (4H, m), 7.15 (2H, d, J 7.8 Hz), 7.07 (1H, br t, J 5.8 Hz), 7.04 (1H, d, J 7.8 Hz), 6.57 (1H, d, J 7.8 Hz), 6.52 (1H, dd, J 6.3, 5.2 Hz), 4.58 (2H, br s), m/z (ES⁺, 70V) 425 [MH^{+].}

### EXAMPLE 7

Z-3-(3-Pyridinyl)-3-(6-{4-[(2-pyridinylamino)methyl]phenoxy}-2-pyridinyl)propenoic Acid

The title compound (440 mg, 68%) was prepared from the compound of Example 5 ((Z)-isomer) in a similar manner to the compound of Example 6. ¹H NMR (d⁶-DMSO) δ 8.55 (1H, dd, J 4.8, 4.7 Hz), 8.49 (1H, d, J 1.9 Hz), 7.95 (1H, m), 7.88 (1H, dd, J 7.4, 7.3 Hz), 7.69-7.66 (1H, m), 7.4, 7.3 (2H, m), 7.30 (2H, d, J 8.6 Hz), 7.08 (1H, d, J 6.8 Hz), 7.04 (1H, d, J 8.6 Hz), 6.99 (2H, d, J 8.1 Hz), 6.54 (1H, s), 6.50-6.45 (2H, m), 4.43 (2H, d, J 4.8 Hz). m/z (ES⁺, 70V) 425 [MH^{+].}

### EXAMPLE 8

3-(3-Pyridinyl)-3-(6-{4-[(2-pyridinylamino)methyl]phenoxy}-2-pyridinyl)propanoic Acid

The compound of Example 7 (130 mg, 0.31 mmol) in ethanol (5 ml) was treated with 10% Pd on carbon (100 mg) and cyclohexadiene (5 ml) then heated under reflux for 18 h. After filtration the reaction mixture was concentrated in vacuo and purified by chromatography (silica; EtOAc/MeOH gradient elution) yielding a colourless oil which was freeze dried from MeOH water to give the title compound (40 mg). ¹H NMR (d⁶-DMSO) δ 8.40 (1H, d, J 2.0 Hz), 8.28 (1H, dd, J 4.7, 4.7 Hz), 7.95 (1H. d,J 6.9 Hz), 7.65 (1H, dd, J 7.8, 7.8 Hz), 7.53 (1H, d, J 8.0 Hz), 7.38-7.32 (3H, m), 7.13 (1H, dd, J 7.9, 7.8 Hz), 7.06-6.99 (3H, m), 6.67 (1H, d, J 8.1 Hz), 6.5 (1H, d, J 8.4 Hz), 6.46 (1H, dd, J 6.8, 5.9 Hz), 4.49 (2H, d, J 4.7 Hz), 4.40 (1H, t, J 7.5 Hz), 2.65 (1H, m), 2.49 (1H, m). m/z (El⁺ 70V) 427 [MH^{+].}

### EXAMPLE 9

3-(2-[4{[(Amino(imino)methyl)amino]methyl}anilino]-6-pyridyl)-3-(4-fluorophenyl)propanoic Acid

Intermediate 10 (0.42 g, 0.63 mmol) was stirred in 1:1 TFA/DCM (10 ml) for 3 h. The solvent was removed by evaporation in vacuo. Purification by chromatography (Silica;15% EtOH, 85% DCM, 1% TFA) gave the title compound (160 mg, 49% as TFA salt). ¹H NMR (DMSO-d⁶) δ 9.10 (1H, br s), 7.90 (1H, br s), 7.68 (2H, d, J 8.0 Hz), 7.50-7.00 (7H, m), 6.62 (1H, d, J 8.0 Hz), 6.55 (1H, d, J 8.0 Hz), 4.45 (1H, t, J 8.0 Hz), 4.28 (2H, d, J 8.0 Hz), 3.21 (1H, dd, J 16.0, 8.0 hH) and 2.91 (1H, dd, J 16.0, 8.0 Hz). m/z (ES⁺, 70V) 408 (MH⁺).

### EXAMPLE 10

t-Butyl-3-(4-fluorophenyl)-3-(2-{4-[(1H-1,3-benzimadazol-2-ylamino)methyl]phenyl}-6-pyridyl-N-oxide)propanoate

To a stirred solution of Intermediate 15 (0.38 g, 1.6 mmol) in DMF (10 ml) under a nitrogen atmosphere at room temperature was added sodium hydride in a single portion (0.064 g, 1.6 mmol, 60% dispersion in oil) and the reaction mixture was stirred for 30 min. Intermediate 3 (0.594 g, 1.5 mmol) was added as a solution in DMF (5 ml). The reaction mixture was heated to 140° C. for 3 h then cooled and poured into saturated sodium hydrogen carbonate solution and extracted twice with DCM. The combined organic fractions were dried (MgSO₄), filtered and the solvent removed by evaporation in vacuo. Purification by chromatography (silica; 10% EtOH/DCM) gave the title compound (0.72 g, 87%). ¹H NMR (CDCl₃) δ 8.00 (1H, br s), 7.32 (2H, dd, J 16.0, 8.0 Hz), 7.20-6.40 (10H, m), 6.62 (2H, d, J 8.0 Hz), 6.55 (1H, d, J 8.0 Hz), 5.43 (1H, t, J 8.0 Hz), 4.58 (2H, br s), 3.75 (1H, dd, J 16.0, 8.0 Hz), 2.92 (1H, dd, J 16.0, 8.0 Hz) and 1.32 (9H, s).

### EXAMPLE 11

3-(4-Fluorophenyl)-3-(2-{4-[(1H-1,3-benzimadazol-2-ylamino)methyl]phenoxy]-6-pyridyl-N-oxide)propanoic Acid Trifluoroacetate Salt

A solution of the compound of Example 10 (0.72 g, 1.3 mmol) in 1:1 TFA/DCM (10 ml) was stirred at room temperature for 72 h and then the solvent was removed by evaporation in vacuo. Purification by chromatography (silica; 1% TFA/25% EtOH/80% DCM) gave the title compound (0.5 g, 74%) ¹H NMR (DMSO-d⁶) δ 12.91 (1H, br s), 9.51 (1H, t, J 6.0 Hz), 7.50-7.30 (8H, m), 7.30-7.10 (5H, m), 6.22 (2H, d, J 9.0 Hz), 5.08 (1H, t, J 8.0 Hz), 4.59 (2H, d, J 5.0 Hz), 3.13 (1H, dd, J 16.0, 8.0 Hz) and 2.98 (1H, dd, J 16.0, 8.0 Hz). m/z (ES⁺, 70V) 499 (MH⁺).

### EXAMPLE 12

3-(4-Fluoropheny)-3-(2-{4-[(1H-1,3-benzimadazol-2-ylamino)methyl]phenoxy}-6-pyridyl)propanoic Acid Trifluoroacetate Salt

To a solution of the compound of Example 11 (0.15 g, 0.3 mmol) in EtOH (10 ml) was added a catalytic amount of palladium on carbon (10%) and excess cyclohexene. The reaction mixture was refluxed for 4 h and then filtered. The solvent was removed by evaporation in vacuo. Purification by chromatography (silica; 1% TFA/20% EtOH/80% DCM) gave the title compound (0.90 g, 62%). ¹H NMR (DMSO-d⁶) δ 7.80-7.70 (2H, m), 7.44 (2H, d, J 8.0 Hz), 7.30-7.20 (2H, m), 7.20-7.15 (1H, m), 7.05-6.95 (5H, m), 6.90-6.80 (1H, m), 6.74 (1H, d, J 8,0 Hz), 4.60 (2H, br s), 4.45 (1H, t, J 8.0 Hz), 3.77 (1H, dd, J 16.0, 8.0 Hz) and 2.95 (1H, dd, J 16.0, 8.0 Hz). m/z (ES⁺, 70V) 483 (MH⁺).

### EXAMPLE 13

Ethyl-3-(6-[hydroxy{4-[(2-pyridinylamino)methyl]phenyl}methyl]-2-pyridinyl)propanoate

Intermediate 20 (7.5 g, 14.85 mmol) in THF (50 ml) was treated with tetrabutylammoniumfluoride (1M in THF, 14.85 ml, 14.85 mmol) and stirred at room temperature for 18 h. The solvent was removed in vacuo and the residue partitioned between EtOAc and water. The organic phase was dried (Na₂SO₄) and concentrated in vacuo. Purification by flash chromatography (silica; EtOAc:hexane 1:1 -2:1) gave the title compound (quantitative). ¹H NMR (CDCl₃) δ 8.08 (1H, d, J 3.8 Hz), 7.51 (1H, t, J 7.7 Hz), 7.38 (1H m), 7.31 (4H, s), 7.08 (1H, d, J 7.6 Hz), 6.91 (1H, d, J 8.0 Hz), 6.58 (1H, dd, J 6.8, 5.2 Hz), 6.34 (1H, d, J 8.4 Hz), 5.68 (1H, s), 4.85 (1H, br t), 4.47 (1H, d, J 5.8 Hz), 4.14 (2H, q, J 7.2 Hz), 3.18 (2H, t, J 7.3 Hz), 2.81 (2H, t, J 7.2 Hz), 1.26 (3H, t, J 7.1 Hz). m/z (ES⁺, 70V) 392 (MH⁺).

### EXAMPLE 14

3-(6-[Hydroxy(4-{[2-pyridinylamino]methyl}phenyl)methyl]-2-pyridinyl)propanoic Acid

The compound of Example 13 (400 mg, 1.02 mmol) and lithium hydroxide (85 mg, 2.04 mmol) were suspended in THF (5 ml) and water (5 ml). After stirring at room temperature for 18 h the THF was removed in vacuo, the residue neutralised with HCl and extracted into DCM and ethanol, dried (Na₂SO₄) and concentrated in vacuo. Purification by radial chromatography (silica; DCM −20% ethanol/DCM) gave the title compound (280 mg). ¹H NMR (DMSO-d⁶) δ 7.62 (1H, br s), 7.43 (1H, t, J 8.8 Hz), 7.5-7.1 (5H, m), 7.10 (1H, d, J 8.0 Hz), 6.60-6.45 (2H, m), 5.95 (1H, br s), 5.72 (1H, br s), 4.45 (2H, br s), 2.47 (2H, t, J 7.0 Hz) and 2.66 (2H, t, J 7.0 Hz); m/z (ES⁺, 70V) 364 (MH⁺).

### EXAMPLE 15

Ethyl-3-(6-{methoxy[4-{(2-pyridinylamino)methyl}phenyl]methyl}2-pyridinyl)propanoate

The compound of Example 13 (1.0 g, 2.49 mmol) was dissolved in THF and cooled to −78° under nitrogen. 1.6M n-Butyllithium (1.71 ml, 2.74 mmol) was added and the reaction stirred for 5 min. Methyl iodide (170 μl, 2.74 mmol) was added and the reaction allowed to warm to room temperature. After quenching with water the product was extracted into DCM, dried (Na₂SO₄) and concentrated in vacuo. ¹H NMR (CDCl₃) δ 8.08 (1H, d, J 4.9 Hz), 7.56 (1H, m), 7.38 (3H, m), 7.28 (3H, m), 7.02 (1H, d, J 7.6 Hz), 6.57 (1H, dd, J 7.0, 5.1 Hz), 6.33 (1H, d, J 8.4 Hz), 5.32 (1H, s), 4.83 (1H, br s), 4.46 (2H, d, J 5.6 Hz), 4.09 (2H, q, J 7.1 Hz), 3.42 (3H, s), 3.20-3.10 (2H, m), 2.80-2.71 (2H, m), 1.20 (3H, t, J 7.1 Hz). m/z (ES⁺, 70V) 406 (MH⁺).

### EXAMPLE 16

3-[6-(Methoxy{4-[(2-pyridinylamino)methyl]phenyl}methyl)-2-pyridinyl]propanoic Acid Trifluoroacetate Salt

The compound of Example 15 (250 mg, 0.62 mmol) was hydrolysed in a similar manner to Example 14. Purification by radial chromatography (silica; DCM, methanol, trifluoacetic acid) gave the title compound (20 mg). ¹H NMR (DMSO-d⁶) δ 7.90 (1H, d, J 7.0 Hz), 7.60 (1H, t, J 7.8 Hz), 7.30-7.20 (6H, m), 7.10 (1H, d, J 7.6 Hz), 6.90 (0.5H, t, J 5.0 Hz), 6.40 (2H, t, J 8.7 Hz), 5.20 (1H, s), 4.40 (2H, s), 3.30 (3H, s), 2.85 (2H, t, J 7.4 Hz) and 2.30 (2H, m). MS (ES⁺) m/e 378 (MH⁺).

### EXAMPLE 17

Ethyl 3-(6-{4-[(2-pyridinylamino)methyl]benzoyl}-2-pyridinyl) propanoate

The compound of Example 13 (1.0 g, 2.56 mmol) and manganese dioxide (2.22 g. 25.6 mmol) in DCM (50 ml) was stirred at room temperature for 18 h. The reaction was filtered and the solution evaporated in vacuo to give the title compound (925 mg). ¹H NMR (CDCl₃) δ 8.13-8.07 (3H, m), 7.85-7.76 (2H, m), 7.48-7.36 (4H, m), 6.64-6.59 (1H, m), 6.39 (1H, d, J 8.4 Hz), 4.95 (1H, br t), 4.62 (2H, d, J 6.0 Hz), 4.07 (2H, q, J 7.1 Hz), 3.18 (2H, t, J 7.3 Hz), 2.83 (2H, t, J 7.4 Hz), 1.17 (3H, t, J 7.1 Hz) m/z (ES⁺, 70V) 390 (MH⁺).

### EXAMPLE 18

3-(6-{4-[(2-Pyridinylamino)methyl]benzoyl}-2-pyridinyl)propanoic Acid

The compound of Example 17 (500 mg, 1.29 mmol) was hydrolysed in a similar manner to Example 14. Chromatography (silica; methanol:DCM 2.5:97.5) gave the title compound (310 mg). ¹H NMR (DMSO-d⁶) δ 8.07-7.91 (4H, m), 7.76 (1H, d, J 7.1 Hz), 7.54 (1H, d, J 7.6 Hz), 7.45 (2H, d, J 8.3 Hz), 7.20-6.50 (1H, m), 7.14 (1H, t, J 5.0 Hz), 6.54-6.45 (2H, m), 4.56 (2H, d, J 5.8 Hz), 3.04 (2H, t, J 7.3 Hz), 2.69 (2H, t, J 7.2 Hz); m/z (ES⁺, 70V) 362 (MH⁺).

### EXAMPLE 19

Ethyl-3-(6-{1-(4-[(2-pyridinylamino)methyl]phenyl)vinyl}-2-pyridinyl)propanoate

Methyltriphenylphosphonium bromide (918 mg, 2.57 mmol) in THF (15 ml) was cooled to 0° and treated with 1.6M butyllithium (1.61 ml, 2.57 mmol). After 30 min a solution of the compound of Example 17 (1.0 g, 2.57 mmol) in THF (10 ml) was added and the reaction warmed to room temperaure then stirred for 18 h. After quenching with water the product was extracted into DCM, dried (Na₂SO₄) and concentrated in vacuo. Chromatography (silica; ether:hexane 1:1) gave the title compound (300 mg). ¹H NMR (CDCl₃) δ 8.10 (1H, m), 7.50 (1H, t, J 7.7 Hz), 7.45-7.26 (5H, m), 7.09 (1H, d, J 7.6 Hz), 7.02 (1H, d, J 7.7 Hz), 6.60 (1H, m), 6.40 (1H, d, J 8.4 Hz), 6.06 (1H, d, J 1.7 HZ), 5.55 (1H, d, J 1.7 Hz), 4.93 (1H, br t), 4.53 (2H, d, J 5.8 Hz), 4.12 (2H, q, J 7.1 Hz), 3.15 (2H, t, J 7.5 Hz), 2.82 (2H, t, J 7.4 Hz), 1.23 (3H, t, J 7.1 Hz). m/z (ES⁺, 70V) 388 (MH⁺).

### EXAMPLE 20

3-(6-{1(4-[(2-Pyridinylamino)methyl]phenyl)vinyl}2-pyridinyl)propanoic Acid

The compound of Example 19 (300 mg, 0.78 mmol) was hydrolysed in a similar manner to Example 14 to give the title compound (183 mg). ¹H NMR (DMSO-d⁶) δ 7.95 (1H, m), 7.67 (1H, t, J 7.7 Hz), 7.50 (1H, m), 7.36-7.27 (4H, m), 7.22 (1H, d, J 7.5 Hz), 7.07 (1H, d, J 7.7 Hz), 6.68 (1H, m), 6.60 (1H, m), 5.96 (1H, s), 5.54 (1H, s), 4.52 (2H, s), 2.97 (2H, t, J 7.3 Hz), 2.65 (2H, t, J 7.3 Hz). m/Z (ES⁺, 70V) 360 (MH⁺).

### EXAMPLE 21

Ethyl-3-(6-[(hydroxyimino)(4-[(2-pyridinylamino)methyl]phenyl) methyl]-2-pyridinyl)propanoate

The compound of Example 17 (500 mg, 1.29 mmol) and hydroxylamino hydrochloride (179 mg, 2.58 mmol) in pyridine were heated at reflux for 18 h. The reaction was quenched with water, extracted into EtOAc, the organic phase washed with HCl then sodium hydrogen carbonate, dried (Na₂SO₄) and concentrated in vacuo to give the title compound as a mixture of isomers (˜1:1) (360 mg). ¹H NMR (CDCl₃) δ 8.62 (1H, m), 8.11 (1H, m), 7.73 (1H, m), 7.59-7.29 (6H, m), 7.15 (1H, t, J 7.1 Hz), 6.61 (1H, t, J 6.2 Hz), 6.42 (1H, dd, J 8.4, 5.3 Hz), 5.27 (0.5H, br t), 5.20 (0.5H, br t), 4.56 (2H, dd, J 7.7, 5.9 Hz), 4.13 (1H, q, J 7.1 Hz), 4.07 (1H, q, J 7.1 Hz), 3.21 (1H, t, J 7.4 Hz), 3.08 (1H, t, J 7.4 Hz), 2.83 (1H, t, J 7.3 Hz), 2.70 (1H, t, J 7.3 Hz), 1.26-1.17 (3H, m). m/Z (ES⁺, 70V) 405 (MH⁺).

### EXAMPLE 22

3-(6-[(Hydroxyimino){4-[(2-pyridinylamino)methyl]phenyl}methyl]-2-pyridinyl)propanoic Acid Trifluoroacetate Salt

The compound of Example 21 (360 mg, 0.89 mmol) was hydrolysed in a similar manner to the compound of Example 14. After 24 h the solvents were removed in vacuo and the residue triturated with methanol, filtered and the filtrate chromatographed (silica; 5% ethanol:1% trifluoroacetic acid, 94% DCM) yielding 180 mg of a mxiture of isomers (1:1) of the title compound.

Isomer A

¹H NMR (DMSO-d⁶) δ 12.10 (1H, br s), 11.40 (1H, s), 7.94 (1H, dd, J 5.8, 1.1 Hz), 7.81 (1H, t, J 7.8 Hz), 7.67 (1H, br s), 7.33 (6H, m), 6.81 (1H, br s), 6.70 (1H, br s), 4.57 (2H, br s), 2.97 (2H, t, J 7.4 Hz), 2.61 (2H, t, J 7.5 Hz).

Isomer B

¹H NMR (DMSO-d⁶) δ 12.05 (1H, br s), 11.60 (1H, s), 7.96 (1H m), 7.74 (1H, t, J 7.8 Hz), 7.59 (1H, m), 7.37-7.31 (4H, m), 7.25 (1H, d, J 7.1 Hz), 6.64 (1H, d, J 8.1 Hz), 6.55 (1H, t, J 6.2 Hz), 4.54 (2H, d, J 4.8 Hz), 2.86 (2H, d, J 7.4 Hz), 2.51 (2H, t, J 7.4 Hz). m/z (ES⁺, 70V) 377 (MH⁺).

### EXAMPLE 23

Methyl 4-[1-(6-{4-[1H-benzoimidazol-2-ylamino)methyl]phenoxy}-1-oxypyridin-2-yl)-2-methoxycarbonylethyl]benzoate

Sodium hydride (60%, 255 mg, 6.14 mmol) was added to Intermediate 15 (1.47 g, 6.14 mmol) in DMF (10 ml)/dioxane (20 ml) and Intermediate 23 (2.2 g, 5.58 mmol) dissolved in dioxane (10 ml) and added. The mixture was heated to 100° for 6 h. The reaction was quenched with water and extracted with DCM (×3), dried with (MgSO₄) and evaporated in vacuo. Purification by column chromatography (silica; DCM −7% methanol) gave the title compound (1.2 g). ¹H NMR δ 8.00 (2H, d, J 8.0 Hz), 7.40 (2H, d, J 8.0 Hz) 7.23-6.97 (8H, m), 5.54-5.49 (1H, t, J 8.0 Hz), 4.58 (2H, s), 3.90 (3H, s), 3.60 (3H, s), 3.38-3.30 (1H, dd, J 16.0, 8.0 Hz), 3.10-3.07 (1H, dd, J 16.0, 8.0 Hz). m/z (ES⁺, 70V) 552 (MH⁺).

### EXAMPLE 24

3-(4-Benzoic acid)-3-(6-{4-[(1H-benimidazol-2-ylamino)methyl]phenoxy}-1 -oxypyridin-2-yl)propanoic Acid

The compound of Example 23 (1.07 g, 1.94 mmol was disslved in 1:1 THF:water (20 ml) and lithium hydroxide monohydrate (0.163 g, 3.83 mmol) was added and the reaction was stirred for 48. Evaporation in vacuo and extraction into DCM/HCl solution yielded the title compound (700 mg) as a cream solid. m/z (ES⁺, 70V) 524 (MH⁺).

### EXAMPLE 25

t-Butyl 3-(6-{4-[6-(4-methoxybenzylamino)pyridin-2-yl]phenoxy}-1-oxypyridin-2-yl)-3-(4-methoxycarbonylphenyl)propanoate

Intermediate 26 (750 mg, 2.45 mmol), t-butyl 4-[1-(6-bromo-1-oxy-pyrid-2-yl)-2-methoxycarbonylethyl]benzoate (prepared in a similar manner to Intermediate 23) (1.06 g, 2.45 mmol) and sodium hydide (60%) (108 mg, 2.70 mmol) was dissolved in DMF (10 ml) and heated at 100° for 19 h. The mixture was extracted into DCM (×3), washed with sodium bicarbonate soluton, dried with (MgSO₄) and evaporated in vacuo. Purification by column chromatography (silica; DCM/5% methanol) gave the title compound (1.0 g). ¹H NMR (CDCl₃) δ 8.01 (2H, d, J 1.2 Hz), 7.98 (2H, d, J 1.8 Hz), 7.44 (3H, m), 7.30 (2H, d, J 4.9 Hz), 7.08 (3H, m), 7.00 (1H, d, J 7.4 Hz), 6.91-6.81 (4H, m), 6.33 (1H, d, J 8.2 Hz), 5.42-5.37 (1H, dd, J 9.0, 7.0 Hz), 4.92 (1H, m), 4.50 (2H, d, J 5.5 Hz), 3.90 (3H, s), 3.80 (3H, s), 3.35-3.28 (1H, dd, J 16.0, 7.0 Hz), 2.98-2.88 (1H, dd, J 16.0, 7.0 Hz) and 1.30 (9H, s). m/z (ES⁺, 70V) 661 (MH⁺).

### EXAMPLE 26

3-(4-Benzoic acid)-3-{6-[4-(6-aminopyridin-2-yl)phenoxy]-1-oxypyridin-2-yl}propanoic Acid Trifluoroacetate Salt

The compound of Example 25 (1 g, 1.51 mmol) and lithium hydroxide (127 mg, 3.03 mmol) were dissolved in a 1:1 mixture of THF and water (20 ml) and stirred for 16 h. This was concentracted in vacuo to which DCM (5 ml) and trifluoroacetic acid (10 ml) were added and stirred for a further 12 h. This was again evaporated in vacuo and purified by chromatography (silica; DCM:EtOAc 9:1) to yield the title compound (800 mg) as a white powder. m/z (ES⁺, 70V) 471 (MH⁺).

### EXAMPLE 27

t-Butyl 3-(4-methoxycarbonylphenyl)-3-{6-[4-(2-pyridinylamino) methyl]phenoxy}-(1 -oxypyridin-2-yl)propanoate

Intermediate 4 (0.46 g, 2.29 mmol) was dissolved in DMF (6 ml) and sodium hydride (0.105 g, 2.52 mmol 60% in mineral oil, pre-washed with hexane) was added. t-butyl 4-[1-(6-bromo-1-oxy-pyrid-2-yl)-2-methoxycarbonylethyl] benzoate (prepared in a similar manner to Intermediate 23) (1.0 g, 2.29 mmol) was dissolved in DMF (6 ml) and added to the reaction mixture which was then heated to 100° under a nitrogen atmosphere for 24 h. The reaction was quenched with saturated NaHCO₃ solution and the crude product extracted into DCM and the solvent removed by evaporation in vacuo and by azeotroping with heptane. Chromatography (silica; 2% EtOH in DCM) gave the title product (0.6 g, 47%). ¹H NMR (CDCl₃) δ 8.08 (1H, d, J 3.9 Hz), 7.97 (2H, d, J 8.4 Hz), 7.44-7.32 (6H, m), 7.11 (1H, t, J 8.0 Hz), 6.98-6.90 (2H, m), 6.78 (1H, dd, J 8.0,1.9 Hz), 6.59 (1H, dd, J 5.2, 0.7 Hz), 6.37 (1H, d, J 8.4 Hz), 5.38-5.35 (1H, m), 5.00 (1H, s), 4.48 (2H, d, J 5.5 Hz), 3.88 (3H, s), 3.28 (1H, dd, J 15.8, 6.7 Hz), 2.92 (1H, dd, J 15.8, 9.2 Hz), 1.28 (9H, s). m/z (ES⁺, 70V) 556 (MH⁺).

### EXAMPLE 28

t-Butyl-3-(4-benzoic acid)-3-{6-[4-(2-pyridylamino)methyl]phenoxy-1-oxy-pyridin-2-yl}propanoate

The compound of Example 27 (0.6 g, 1.08 mmol) was dissolved in THF (5 ml) and H₂O (5 ml). LiOH.H₂O (0.1814 g, 4.32 mmol) was added and the reaction mixture stirred overnight at room temperature before being acidified with 1M HCl. The organics were extracted into DCM and concentrated in vacuo to give the title compound (0.26 g, 44%). ¹H NMR (CDCl₃) δ 7.86-7.68 (3H, m), 7.38-7.14 (6H, m), 7.01 (3H, d, J 8.0 Hz), 6.86 (1H, d, J 8.0 Hz), 6.78 (1H, t, J 5.0 Hz), 6.65 (1H, d, J 8.0 Hz), 5.23 (1H, m), 4.53 (2H, d, J 5.5 Hz), 3.20-2.94 (2H, m), 1.39-1.24 (9H, m).

### EXAMPLE 29

3-(4-Benzoic acid)-3-(6-{4-[(2-1pyridylamino)methyl]phenoxy}-1-oxypyridin-6-yl)propanoic Acid Trifluoroacetate Salt

The compound of Example 28 was dissolved in TFA (5 ml) and DCM (5 ml). The reaction mixture was stirred overnight and the crude product isolated by evaporation in vacuo. Radial chromatography (5% MeOH in DCM then 10% MeOH in DCM) gave the title compound which was freeze dried to give a white solid (0.14 g, 62%). ¹H NMR (DMSO-d⁶) δ 8.02 (1H, d, J 4.8 Hz), 7.92 (2H, d, J 8.3 Hz), 7.74 (1H, t, J 7.1 Hz), 7.51 (3H, d, J 8.3 Hz), 7.46-7.41 (3H, m), 7.24 (1H, dd, J 8.1, 1.6 Hz), 6.97 (2H, d, J 8.6 Hz), 6.89 (1H, d, J 8.3 Hz), 6.78 (1H, t, J 6.1 Hz), 5.23 (1H, t, J 7.9 Hz), 4.56 (2H, s), 3.28 (1H, dd, J 16.6, 7.8 Hz), 3.12 (1H, dd, J 16.6, 8.1 Hz).

The following assays may be used to determine the ability of compounds according to the invention to inhibit αᵥβ₃ and αᵥβ₅ function.

αᵥβ₃-Dependent Direct Binding Assay

96 Well NUNC immunoplates were coated overnight with a non-blocking anti-β3 monoclonal antibody at 2 μg/ml in Dulbecco's phosphate buffered saline (PBS) and subsequently blocked with 5% 9w/v)BSA in PBS (Sigma, fraction V) for 60 min. at room temperature. After washing in Tris-buffered saline (TBS: 20 mM Tris/150 mM NaCl, pH 7.5), plates then received 100 μl of a lysate prepared fromn JY cells and were incubated for 3 h at room temperature. The lysate was made by lysing JY B-lymphoblastoid cells at 5×10⁷ cells were ml in TBS containing 1 mM MnCl₂,1% (w/v) BSA/0.1% (vb/v) Tween 20 and were incubated for a further 2 hours at room temperature. Inhibitors were titrated into the fibronectin prior to addition to plates. After washing, streptavidin-peroxidase (Amersham) at 1:500 in TBS/1% (w/v) BSA/0.1% (v/v)Tween 20 was added and plates incubated for 1 h at room temperature. Finally 100 μl TMB substrate was added and Absorbance (630 nm) measured after 10-15 minutes. IC₅₀ values for inhibition of adhesion were calculated on the Activity Base curve fitting programme.

αᵥβ₃-Dependent Cell Adhesion Assay

This was a modification of a published method [Stupack et al., Exp,. Cell. Tes. 203, 443-448 (1992)] and employed the JY cell line. These cells are maintained in RPMI 1640+10% FCS +2 mM L-glutamine and, when used for assay, were washed in assat medium (RPMI 1640+10% FCS), suspended at 4×10⁶/ml in the same medium and pretreated with a blocking monoclonal antibody to CD18 (6.5E, F(ab′)₂ fragment) for 10 min at room temperature. 96 Well NUNC immunoplates were coated with 100 μl 2.5 uk/μl human vitronectin in PBS per well for 2 h at 37° C.; they were then washed 2× in PBS and blocked with 1% (w/v) BSA in PBS for 60 min at room temperature and washed 2× more in PBS. 2×1⁻⁵ JY per well were added to wells containing compounds serially titrated across the plate and, finally, phorbol-12-myristate-13-acetate at 10 ng/ml was added in a final volume of 200 μl. After incubation at 37° C. for 30 min, non-adherent cells were removed by washing 3× in assay medium, adherent cells were fixed in methanol and stained with 0.25% (W/v) Rose Bengal in PBS for 5 min, unbound dye was removed by 3 further washes in PBS and cell-bound dye was released with 1:1 PBS:ethanol. Absorbance at 570 nm was then measured. IC₅₀ values for inhibition of adhesion were calculated as described above for the direct binding assay.

αᵥβ₅-Dependent Cell Adhesion Assay

This assay was based on a published method [Koivunen et al, J. Bio. Chem. 268, 20205-20210 (1993)] and employed the human colon adenocarcinoma cell line HT-29. HT-29 Cells were routinely maintained in DMEM +10% FCS +2 mM L-glutamine and were removed from flasks using trypsin/EDTA, washed 2× in assay medium and suspended at 4×10⁶/ml in the same medium. The cells were allowed to ‘rest’ for 15 min. at room temperature before being added (2×10⁵/well) to wells containing compounds serially titrated across the plate in a final volume of 200 μl. The 96 well NUNC immunoplates had been coated with human vit ronectin as described above for the αᵥβ₃ assay. After incubation at 37° C. for 60 min, adhesion was assessed as described above for the αᵥβ₃ assay.

In the above assays the preferred compounds of the invention generally have IC₅₀ values of 1 μM and below.

1. A compound of formula (1):
Ar—X¹—Ar¹—Z—R (1)
wherein:
(1) Ar is a group R^{1a}[N(R²)]_{q}L¹Ar²— in which:
R^{1a} is a nitrogen base and q is zero or the integer one;
R² is a hydrogen atom or an optionally substituted aliphatic, heteroaliphatic, cycloaliphatic, polycycloaliphatic, heterocycloaliphatic, heteropolycycloalphatic, aromatic or heteroaromatic group;
L¹ is a covalent bond or a —[C(R³)(R⁴)]ₙ— [where R³ and R⁴, which may be the same or different, is each a hydrogen atom or a straight or branched alkyl group or a hydroxyl group and n is the integer one or two], —C(O)—, —C(S)—, —S(O)—, —S(O)₂—, —P(O)—, —P(O)(OR^{a})— [where R^{a} is a hydrogen atom or a straight or branched C₁₋₆alkyl group] or —P(O)(OR^{a})O— group; and
Ar² is an optionally substituted six-membered 1,4-arylene or 1,4-heteroarylene ring; or
(2) Ar is a bicyclic ring: 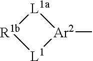
in which R^{1b} is a nitrogen base, L¹ and Ar² are as just defined and —L^{1a}— is a covalent bond a —(CH₂)₃— group or a group L¹ as just defined;
X¹ is an —O— or —S— atom or a group selected from —C(O)—, —C(S)—, —S(O)—, —S(O)₂—, —C(R⁵)(R⁶)— {where R⁵ is a hydrogen atom or an optionally substituted straight or branched alkyl group and R⁶ is a hydrogen or halogen atom or a straight or branched alkyl, haloalkyl, haloalkoxy, alkylthio, aromatic, heteroaromatic, or —(Alk¹)ₘR⁷ group [in which Alk¹ is a C₁₋₃alkylene chain, m is zero or the integer 1 and R⁷ is a —OH, —SH, —NO₂, —CN, —CO₂H, —CO₂R⁸ (where R⁸ is an optionally substituted straight or branched C₁₋₆alkyl group), —OR⁸, —SO₃H, —SOR⁸, —SO₂R⁸, —SO₃R⁸, —OCO₂R⁸, —C(O)H, —C(O)R⁸, —OC(O)R⁸, —C(S)R⁸, —NR⁹R¹⁰ (where R⁹ and R¹⁰, which may be the same or different is each a hydrogen atom or a straight or branched alkyl group), —C(O)N(R⁹)(R¹⁰), —OC(O)N(R⁹)(R¹⁰), —N(R⁹)C(O)R¹⁰, —CSN(R⁹)(R¹⁰), —N(R⁹)C(S)R¹⁰, —SO₂N(R⁹)(R¹⁰), —N(R⁹)SO₂R¹⁰, —N(R⁹)C(O)N(R¹⁰)(R¹¹) [where R¹¹ is a hydrogen atom or a straight or branched alkyl group], —N(R⁹)C(S)N(R¹⁰)(R¹¹), —N(R⁹)SO₂N(R¹⁰)(R¹¹), aromatic or hetero-aromatic group]}, —C(═NOH)—, —C(═CR⁵R⁶)— or —N(R⁵)—;
Z is a group —CH(R¹³)CH₂— [in which R¹³ is R^{13a} or Alk^{1a}R^{13a}, R^{13a} is a hydrogen atom or an optionally substituted aliphatic, cycloaliphatic, heteroaliphatic, heterocycloaliphatic, aromatic or heteroaromatic group, and Alk^{1a} is a C₁₋₃alkylene chain optionally substituted with one, two, three or more halogen atoms or straight or branched alkyl, haloalkyl, alkoxy, haloalkoxy, alkylthio, aromatic or heteroaromatic groups], —C(R^{12a})(R¹³)—CH(R^{12b})— [in which R^{12a} and R^{12b} together with the carbon atoms to which they are attached form a C₃₋₇cycloalkyl group] or —C(R¹³)═CH—;
R is a carboxylic acid (—CO₂H) or a derivative or biostere thereof;
Ar¹ is an optionally substituted heterocycle of formula 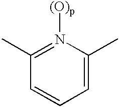
wherein p is zero or the integer 1;
and the salts, solvates, hydrates and N-oxides thereof.

2. A compound according to claim 1 in which R is a —CO₂H group.

3. A compound according to claim 1 in which X¹ is a —O— or —S— atom or —NH—, —N(CH₃)—, —C(R⁵)(R)⁶—, —(C(═CR⁵R⁶)— or —C(═NOH)—group.

4. A compound according to claim 3 in which X¹ is a —CH₂—, —CH(OH)— or —CH(OCH₃)—group.

5. A compound according to claim 1 in which Ar is a group R^{1a}[N(R²)]_{q}L¹Ar² in which R² is a hydrogen atom.

6. A compound according to claim 5 in which Ar² is an optionally substituted 1,4-phenyl, 2,5-pyridyl or 2,5-pyrimidinyl group.

7. A compound according to claim 1 in which Z is a —CH(R¹³)CH₂— or —C(R¹³)═CH—group.

8. A compound according to claim 7 in which R¹³ is an optionally substituted aromatic or heteroaromatic group.

9. A compound according to claim 8 in which R¹³ is an optionally substituted phenyl or five- or six-membered heteroaromatic group.

10. A compound according to claim 9 in which R¹³ is an optionally substituted pyridyl, pyrimidinyl or pyridine-N-oxide group.

11. A compound according to claim 1 in which R^{1a} is a R¹⁴R¹⁵NC(X²)— or R¹⁵X(═NR¹⁴)— group.

12. A compound according to claim 11 in which R^{1a} is a H₂NC(═NH)—group.

13. A compound according to claim 1 in which R^{1a} is an optionally substituted five- to ten-membered nitrogen containing heterocycloaliphatic group or a five- to ten-membered nitrogen containing heteroaromatic group.

14. A compound according to claim 13 in which R^{1a} is an optionally substituted imidazolinyl, imidazolyl, pyridyl, benzoimidazolyl, tetrahydropyrimidinyl, tetrahydro-[1,8]-naphthyridinyl, [1,8]-naphthyridinyl or triazolyl group.

15. A compound according to claim 1 in which q is zero and L¹ is a covalent bond.

16. A compound according to claim 1 in which q is zero and L¹ is a —C(R³)(R⁴)— group.

17. A compound according to claim 16 in which L¹ is a —CH₂— or —CHOH— group.

18. A compound according to claim 1 in which q is the integer 1 and L¹ is a —C(O)— or —C(R³)(R⁴)— group.

19. A compound according to claim 18 in which L¹ is a —CH₂— or —CHOH— group.

20. A compound according to claim 1 in which q is the integer 1 and L¹ is a covalent bond.

21. A compound which is
3-(4-Fluorophenyl)-3-(2-{4-[(2-pyridinylamino)methyl]phenoxy}-pyrid-6-yl-N-oxide)propanoic acid trifluoroacetic acid salt;
3-(3-Pyridinyl)-3-(6-{4-[(2-pyridinylamino)methyl]phenyl}-2-pyridinyl) propanoic acid;
3-(4-Fluorophenyl)-3-(2-{4-[(1H-1,3-benzimadazol-2-ylamino)methyl]phenoxy]-6-pyridyl-N-oxide)propanoic acid rifluoroacetate salt;
3-(4-Benzoic acid)-3-(6-{4-[(1H-benimidazol-2-ylamino)methyl]phenoxy}-1-oxypyridin-2-yl)propanoic acid;
3-(4-Benzoic acid)-3-{6-[4-(6-aminopyridin-2-yl)phenoxy]-1-oxypyridin-2-yl}propanoic acid trifluoroacetate salt;
3-(4-Benzoic acid)-3-(6-{4-[(2-pyridylamino)methyl]phenoxy}-1-oxypyridin-6-yl)propanoic acid trifluoroacetate salt;
and the salts, solvtes, hydrtes and N-oxides thereof.

22. A pharmaceutical composition comprising a compound according to claim 1 together with one or more pharmaceutically acceptable carriers, excipients or diluents.
